(19) United States
(12) Patent Application Publication
(10) Pub. No.: US 2005/0004476 A1
(43) Pub. Date: Jan. 6, 2005

(54) Method and apparatus for detecting ischemia

(76) Inventors: Saeed Payvar, Houston, TX (US); Samuel Ward Casscells III, Houston, TX (US)

Correspondence Address: TIM L. BURGESS, P.C., 402 OAK LANE, HOUSTON, TX, 77024, US

(21) Appl. No.: 10/857,603

(22) Filed: May 28, 2004

Related U.S. Application Data
(60) Provisional application No. 60/473,771, filed on May 28, 2003.

(51) Int. Cl. A61B 5/02

(52) U.S. Cl. 600/481; 600/549

(57) ABSTRACT

The current invention includes methods to monitor and detect myocardial ischemia/infarction using apical regional temperature and/or coronary sinus blood temperature, and comparing those measurements to regional baselines as well as the core body temperature. Methods for combining such temperature measurements with a broad series of other indicators of myocardial ischemia/infarction are explained. This invention also includes description of devices that put the above described method in use, as well as includes means to provide the patient and/or his health-care provider with easily accessible information leading to timely initiated therapy, and also means to communicate with other devices that provide such early treatment.

### CROSS REFERENCE TO RELATED APPLICATIONS

This application claims benefit of priority to U.S. Provisional Application Ser. No. 60/473,771, filed May 28, 2003.

### STATEMENT REGARDING FEDERALLY SPONSERED RESEARCH OR DEVELOPEMENT

The U.S. government may own rights in the present invention pursuant to grant number DAMD 17-01-2-0047 from the U.S. Army Medical Research Acquisition Activity.

### BACKGROUND OF THE INVENTION

1. Field of the Invention

The invention includes devices and methods for detection, continuous monitoring and treatment of myocardial ischemia and infarction.

2. Description of the Background Art

Examples of situations in which this invention is specifically useful are in patients with Coronary Heart Disease (CHD) who are at higher risk of Silent Myocardial Ischemia (SMI) and/or myocardial infarction and in other patients at risk of myocardial ischemia in whom another disease or condition would make the detection of such ischemia difficult or delayed.

There are 10 million people in the United States that have CHD. These patients are at risk of myocardial ischemia, i.e. lack of blood supply to different regions of the heart muscle. This usually causes angina (discomfort in the chest) and this, together with evidence coming from laboratory and other investigational methods, leads to the detection of the ischemia. However, there are situations in which detection of myocardial ischemia is delayed because of nonexistence of angina, or inaccurate or even impossible because of limitations in the currently available diagnostic tools or the expenses incurred for their use. This in turn compromises the well-being or even the survival of the patient.

Examples of such difficulties in the diagnosis of ischemia include such diagnosis in patients at risk of silent myocardial ischemia, which is described in detail below. Other examples include such diagnosis in CHD patients who:
a) Have left bundle branch block (LBBB). LBBB is an abnormality of heart's electrical activity, which would mask early signs of ischemia, hence making diagnosis of ischemia difficult.
b) Have a pacemaker. In these patients the electrical activity of the pacemaker would mask signs of ischemia.
c) Are unconscious.
d) Cannot communicate their pain.

Patients with CHD are at risk of myocardial ischemia (lack of blood supply to heart muscle cells) and/or infarction (the death of the heart muscle because of ischemic causes), which usually manifest themselves with angina. However, in 40% of these patients, episodes of ischemia are asymptomatic (Silent Myocardial Ischemia, SMI) and close to one third of infarctions are silent as well. Silent ischemia might progress to myocardial infarction and myocardial infarction is the most common cause of heart failure and/or cardiac death among these patients. Of 708 myocardial infarctions among 5127 participants in the Framingham Study, more than 25% were diagnosed during routine biennial electrocardiography (EKG) examination, of which almost 50% were silent and the others caused atypical symptoms. While recurrence of infarctions was more likely in women with recognized infarctions than in women with unrecognized infarctions, such distinction was not present in men. Such unrecognized infarctions were as likely as recognized ones to cause death, heart failure, or strokes.

Astonishingly, SMI might exist with angina or with angina treatment. Approximately, 20-30% of patients with CAD have SMI during usual daily activities. Moreover, an estimated 80% of ischemic episodes in patients with a history of angina are asymptomatic. Among patients whose symptoms are controlled with angina suppressing medications, up to 40% continue to have SMI.

If detected, SMI may warrant suppressive therapy. There is not enough evidence to determine whether or not total suppression of SMI is required to improve clinical outcome and reduce cardiac events. However, it has been suggested that therapy to reduce ischemia may eliminate or at least reduce symptomatic and silent ischemia. Modes of therapy include beta-blocker medications, calcium channel blockers, nitrates, and myocardial revascularization. Combination drug therapy may achieve total suppression of ischemia in at least 75% of patients, which is similar to that achieved by myocardial revascularisation. Evidence is however lacking whether there is a need to reassess the presence and extent of silent ischemia during therapy, for such assessment would not have the specificity and sensitivity that is required for such fine-tuning. EKG ischemia guided strategy has been used achieve optimal regimens.

Current techniques for the detection of SMI are only moderately sensitive and even less specific, especially in certain subgroups of patients. “Sensitive” in epidemiology describes an indicator that is strong in ruling out a disease or condition, and “specific” describes an indicator that is sure to detect a disease when the person is in fact affected. Exercise treadmill EKG testing is the preferred screening technique, but it is only moderately sensitive and has an unacceptably high false positive rate, particularly in the young and in women but is faulsley negative4 in elderly. Exercise treadmill testing is usually substituted by pharmacologic testing in the elderly, because of the underlying conditions such as musculoskeletal abnormalities, arthritis or neurological deficit, as these conditions keep the patient from exercising long enough to become ischemic. SMI suspected in this way must be. confirmed with radionuclide imaging techniques (perfusion scintigraphy or exercise ventriculography) or stress echocardiography. Further, EKG testing is very insensitive for detecting SMI in the apex area of the right ventricle, for there the myocardium is much thinner (only about 3 mm thick). This insensitivity is particularly critical because the apex is the watershed area of the entire heart making it more susceptible to risk of ischemia than the rest of the heart.

Apart from relative lack of sensitivity and specificity, these mentioned diagnostic techniques pose other challenges. Continuous ambulatory EKG monitoring requires hospitalization, is uncomfortable to the patient, difficult in a home setting, and is difficult to interpret because of the large number of artifacts. Routine exercise EKG testing is less accurate in cases with Left Bundle Branch Block (LBBB) and is expensive to do on a regular basis and is radioactive. Radionuclide scintigraphy is expensive and difficult to do on a routine basis.

### SUMMARY OF THE INVENTION

The current invention, among other advantages that it possesses, is directed toward SMI, a specific disease state not before targeted for continuous monitoring, detection and treatment, and to myocardial infarction.

In our investigations, we have made a number of surprising discoveries leading to new ways and devices for monitoring and detecting SMI and myocardial infarction to allow and provide treatment of the patient to prolong life.

We have discovered a significant increase in the temperature in the coronary sinus of the heart is associated with ischemia; that this rise precedes electrocardiographic changes and development of symptoms, allowing early warnings that the conventional methods do not; and that a significant fall in temperature in the coronary sinus of the heart below baseline is associated with the removal of ischemia.

We have invented the following new methods for detecting SMI based on temperature and the waveform of the temperature sensor located in the coronary sinus:

(A1) Placing a temperature sensor in the coronary sinus of a heart and taking a blood core temperature (T_{C}) elsewhere, measuring temperature in the coronary sinus (T_{CS}) sensed by said sensor in the coronary sinus and determining if T_{CS} increases relative to T_{C}. The most accurate core temperature for this invention is from the left or right coronary arteries, but this temperature is restricted to in hospital settings. For chronic indwelling catheters and leads, core temperature is best from the right atrium or right ventricle.

(A2) Placing a temperature sensor in the coronary sinus of a heart after taking a baseline temperature (T-baseline) in the right atrium of the heart with said sensor, measuring temperature in the coronary sinus (T_{CS}) sensed by said sensor in the coronary sinus and determining if TCS increases relative to T-baseline.

(A3) Placing a temperature sensor in the coronary sinus of a heart and a temperature sensor in the right atrium of a heart, measuring temperature in the coronary sinus (T_{CS}) sensed by said sensor in the coronary sinus and measuring temperature in the right atrium (T_{RA}) sensed by said sensor in the right atrium and determining if T_{CS} increases relative to T_{RA}.

(A4) Placing a temperature sensor in the coronary sinus of a heart, measuring the waveforms of temperature in the coronary sinus sensed by said sensor in the coronary sinus relative to a core blood temperature baseline and determining if the average of the waveforms increases.

In respect to method (A4) an embodiment is characterized by determining if the average increased waveform is characterized by a rapid slope of increase slowing to a plateau.

In respect to method (A4) an embodiment is characterized in that said average is the mean of the oscillation of the waveform.

In respect to method (A4) an embodiment is characterized in that said average is the size of the area of the curve under the waveform.

In respect to method (A4) an embodiment is one in which said core temperature baseline is taken by said sensor in the right atrium of the heart before placing the sensor in the coronary sinus.

In respect to method (A4) an embodiment further comprises placing a temperature sensor in the right atrium of the heart and measuring temperature in the right atrium (T_{RA}) sensed by said sensor in the right atrium to establish baseline core temperature.

In respect to method (A4) an embodiment further comprises determining the size and/or severity of a region of ischemia by determining the increase of average of the waveforms before cessation of an event of ischemia.

In respect to method (A4) an embodiment further comprises determining cessation of an event of ischemia by determining when the waveform average decreases after said increase, to values below pre-ischemic values.

In respect to method (A4) an embodiment further comprises determining cessation of an event of ischemia by determining that the slope of decrease of the average of the waveform is sharper than the slope of increase of the waveform.

(A5) Placing a temperature sensor in the coronary sinus of a heart, measuring the waveforms of temperature in the coronary sinus (T_{CS}) sensed by said sensor in the coronary sinus relative to a core blood temperature baseline and determining if the frequency of the waveforms increases.

In respect to method (A5) an embodiment further comprises determining the size and/or severity of a region of ischemia by determining an increase of frequency the waveforms before cessation of an event of ischemia.

In respect to method (A5) an embodiment further comprises determining cessation of an event of ischemia by determining when the waveform frequency decreases after said increase to pre-ischemic values.

In respect to method (A5) an embodiment is one in which said core temperature baseline is taken by said sensor in the right atrium of the heart before placing the sensor in the coronary sinus.

In respect to method (A5) an embodiment further comprises placing a temperature sensor in the right atrium of the heart and measuring temperature in the right atrium (T_{RA}) sensed by said sensor in the right atrium to establish baseline core temperature.

(A6) Placing a temperature sensor in the coronary sinus of a heart, measuring the waveforms of temperature in the coronary sinus (T_{CS}) sensed by said sensor in the coronary sinus relative to a core blood temperature baseline, and determining if the amplitude of the waveforms decreases.

In respect to method (A6) an embodiment further comprises determining the size and/or severity of a region of ischemia by determining the decrease of amplitude of the waveforms.

In respect to method (A6) an embodiment is one in which said core temperature baseline is taken by said sensor in the right atrium of the heart before placing the sensor in the coronary sinus.

In respect to method (A6) an embodiment further comprises placing a temperature sensor in the right atrium of the heart and measuring temperature in the right atrium (T_{RA}) sensed by said sensor in the right atrium to establish baseline core temperature.

We have discovered that the temperature of the myocardium of the apex, unlike that of the temperature in the coronary sinus, decreases during ischemia. We have invented the following new methods for detecting SMI based on temperature and the waveform of the temperature sensor attached to the wall of the apex of the heart:

(B1) Attaching a temperature sensor in the wall of the apex of a heart, taking a blood core temperature (T_{C}) elsewhere, measuring temperature of the apex (T-apex) sensed by said sensor in said wall and determining if T-apex decreases relative to T_{C}.

(B2) Attaching a temperature sensor in the wall of the apex of a heart after taking a baseline temperature (T-baseline) in the right atrium of the heart with said sensor, measuring temperature of the apex (T-apex) sensed by said sensor in said wall and determining if T-apex decreases relative to T-baseline.

(B3) Attaching a temperature sensor in the wall of the apex of a heart and a temperature sensor in the right atrium of a heart, measuring temperature of the apex (T-apex) sensed by said sensor in said wall and measuring temperature in the right atrium (T_{RA}) sensed by said sensor in the right atrium and determining if T-apex decreases relative to TRA.

We have invented the following new methods for detecting SMI based on temperature and the waveform of the temperature sensor located in the coronary sinus temperature and on the waveform of the temperature sensor attached to the wall of the apex:

(C1) Placing a temperature sensor in the coronary sinus of a heart, taking a blood core temperature (T_{C}) elsewhere, attaching a temperature sensor in the wall of the apex of a heart, measuring temperature in the coronary sinus (T_{CS}) sensed by said sensor in the coronary sinus, measuring temperature of the apex (T-apex) sensed by said sensor in said wall, and determining if the ratio of T_{CS} to T-apex increased.

(C2) Placing a temperature sensor in the coronary sinus of a heart after taking a baseline temperature (T-baseline) in the right atrium of the heart with said sensor, attaching a temperature sensor in the wall of the apex of a heart, measuring temperature in the coronary sinus (T_{CS}) sensed by said sensor in the coronary sinus, measuring temperature of the apex (T-apex) sensed by said sensor in said wall, and determining if and determining if the ratio of T_{CS} to T-apex increases.

(C3) Placing a temperature sensor in the coronary sinus of a heart and a temperature sensor in the right atrium of a heart, attaching a temperature sensor in the wall of the apex of a heart, measuring temperature in the coronary sinus (T_{CS}) sensed by said sensor in the coronary sinus, measuring temperature of the apex (T-apex) sensed by said sensor in said wall, and determining if the ratio of T_{CS} to T-apex increases.

The use of methods (A1)-(A6) involving a temperature sensor in the coronary sinus of a heart may be advantageous in some respects compared to methods (B1)-(B3) involving attaching a temperature sensor in the wall of the apex of a heart, in that T_{CS} may show larger changes in response to ischemia and the temperature waveforms are more closely related to blood flow. On the other hand T-apex may be more responsive to ischemia in a variety of regions of the heart, since the right ventricular apex is the watershed of heart, whereas Tcs changes may represent a comparatively smaller area of the heart. An advantage of T-apex compared to T_{CS} is the former allows an electrogram as well. Methods (C1)-(C3) may give a higher level of sensitivity and selectivity in that they monitor both the coronary sinus and the apex. Either an increase in T_{CS} relative to T-apex or a decrease of T-apex relative to T_{CS}, or a combination of an increase in T_{CS} and a decrease of T-apex will signal ischemia.

In addition we have discovered a novel method detecting rhythm abnormality of the heart comprising placing a temperature sensor in the coronary sinus of a heart, measuring the waveforms of temperature in the coronary sinus (T_{CS}) sensed by said sensor in the coronary sinus relative to a core blood temperature baseline, and determining if the waveforms of temperature in the coronary sinus is atypical for waveforms of temperature in the normal heart.

In addition we have discovered a novel method of tracing pressure in coronary arteries comprising placing a temperature sensor in the coronary sinus of a heart, measuring the waveforms of temperature in the coronary sinus (T_{CS}) sensed by said sensor in the coronary sinus relative to a core blood temperature baseline, and ascertaining the phase of said waveforms.

In addition we have discovered a novel method of determining blood flow in coronary arteries comprising placing a temperature sensor in the coronary sinus of a heart, measuring the waveforms of temperature in the coronary sinus (T_{CS}) sensed by said sensor in the coronary sinus relative to a core blood temperature baseline, and ascertaining the shape of said waveforms.

In an aspect of the invention, the devices of the devices of the invention for detecting cardiac ischemia are connected with a pacemaker, a defibrillator, a ventricular assist device or a controller for a medical infusion pump as a means of furnishing immediate therapy on detection of ischemia. The inclusion of an ischemia detecting device of this invention with an algorithm for pacing allows the pacing to be maintained at a base level which paces for survival instead of pacing the heart for increased work, either in connection with a pacemaker or a defibrillator.

One specific design of this invention is a device that detects that region of the heart which is lacking blood supply (i.e. is ischemic). This specific device is useful in locating the culprit lesion in situations in which there are several lesions in the heart's arterial supply tree and detecting the one that is causing more ischemia is of prime importance.

Other specific designs of the device are explained, and these will have better accuracy by correcting for possible confounding factors using one or more of physiologic indicators. In other specific designs, the usefulness of the device goes beyond the detection of ischemia and covers detection and monitoring for a variety of other diseases of the heart. Still in another set of designs, the usefulness of the device goes beyond detection and monitoring for ischemia and covers methods to prevent and/or treat such ischemia when it would develop.

The invention contemplates a method, software, and devices for indicating myocardial ischemia in patients with coronary heart disease recording the attributes of temperature changes in the apex (T-apex) (i.e. the attributes of T-apex changes) and/or coronary sinus (CS) (i.e. the attributes of T_{CS} changes) and/or gradient of those with the core (C) temperature as measured in the right atrium [i.e. the attributes of the changes of (T_{CS}−T_{C}) and (T-apex−T_{C})].

In accordance with this invention, a method of analyzing the above-said temperature readings in order to detect myocardial ischemia comprises: presetting the specificity and sensitivity, subsequently obtaining T-apex and/or T_{CS} and T_{C} in short intervals and establishing each region's own baseline temperature variations as well as the respective gradients of with one another, obtaining the status of other factors indicative of myocardial ischemia and patient's current physical and environmental status, and determining whether the patient's current RV-A and/or CS regional temperatures and/or gradient of those temperatures with core body temperature (collectively named static measures), or their variations (collectively named dynamic measures) might fit predetermined criteria for myocardial ischemia, taking into consideration the preset sensitivity and specificity, the status of other factors indicative of myocardial ischemia and patient's current physical and environmental status.

Another preferred embodiment of the method of the invention involves means of combining the above-said attributes of temperature changes with other factors indicative of myocardial ischemia, including decreased coronary sinus pH, decreased coronary sinus pO₂, increased coronary sinus pCO₂, increased coronary sinus lactate, increased ratio of lactate to pyruvate in the coronary sinus, increased ratio of the reduced form of nicotine amide adenine dinucleotide (NADH) to nicotine amide adenine dinucleotide (NAD⁺) in the coronary sinus, increased ratio of the reduced form of nicotinamine-adenine dinucleotide phosphate (NADPH) to nicotinamine-adenine dinucleotide phosphate (NADPH) in the coronary sinus, increased ST segment, decreased ST segment, ventricular tachycardia, T wave changes, QRS changes, decreased patient activity, increased respiratory rate, decreased transthoracic impedance, decreased cardiac output, increased pulmonary artery diastolic pressure, increased myocardial creatinine kinase, increased troponin, and changed myocardial wall motion.

Another preferred embodiment of the method of the invention involves means to alarm the patient and/or the patient's healthcare professional, family, friends, emergency medical service, call 911 or tale other alerting action. Such embodiment comprises: presetting the alarming route for the warning system, determining if the above-said criteria for myocardial ischemia are met, and warning the patient and/or the patient's healthcare provider using the predetermined route.

Another preferred embodiment of the method of the invention involves means to initiate regional and/or systemic therapy. Therapeutic means include modifying parameters of pacemakers, defibrillators, or mechanical heart pump assist devices, or administering substances in small doses for regional effect or in large doses for systemic effect, through internally located (i.e. implanted) controlled substance release pumps or externally located infusion pumps. Said substances include at least one of anticoagulation or antithrombotic, thrombolitic or antiarrhythmitic medications, heart rhythm modifying agents, coronary/systemic vasodilators, analgesics and anti-inflammatory medications. Such embodiment comprises: presetting the therapeutic means, determining if the above-said criteria for myocardial ischemia are met, and initiating therapy though the predetermined therapeutic means.

The invention embodies apparatus and software for the implementation of invention. A device useful in the present invention for monitoring for myocardial ischemia preferably includes two parts: 1) implantable central processing unit with capabilities to communicate alarms or information with the patient, the patient's health caregiver, or other monitoring and therapeutic medical devices through wireless means; 2) three implantable temperature sensors located in the right atrium (RA), apex, suitably the right apex, and CS.

### DESCRIPTION OF THE DRAWINGS

FIG. 1 is a posterior inferior view of an anatomical representation of a human heart showing the coronary sinus and veins that drain into it.

FIG. 2 is an anterior superior view of an anatomical representation of a human heart showing the veins that drain into the coronary sinus

FIG. 3 is a diagrammatic representation of the human heart sectioned to reveal the interior chambers of the heart and the opening (ostium) of the coronary sinus into the right atrium of the coronary sinus and its relation to other parts of heart.

FIG. 4 is a depiction of a configuration of a device in accordance this invention.

FIG. 5 is a diagrammatic depiction of a use of a system in accordance with this invention with placement of leads inside the heart.

FIG. 6 is a diagrammatic depiction of a use of a system in accordance with this invention with a single coronary sinus lead.

FIG. 7 is a diagrammatic depiction of a use of a system in accordance with this invention with a single right ventricular apex lead.

FIG. 8 is a diagrammatic depiction of a use of a system in accordance with this invention with leads in both right ventricular apex and coronary sinus.

FIG. 9 is a flow chart schematically showing a sequence of operations performable by an algorithm in accordance with this invention.

FIG. 10 is a continuation of the chart of FIG. 9.

FIG. 11 is a diagram of the circuitry for a device in accordance with this invention.

FIG. 12 is a fluoroscopic image showing the location of temperature sensors in the coronary sinus and right ventricular apex of a male 28 Kg mongrel dog using a surgical approach for placement in an acute experiment.

FIG. 13 is a picture showing the location of temperature sensor at the end of the first turn of the screw of an active fixation screw-in pacing lead, and the wire connected to the temperature sensor for signal transmission.

FIG. 14 is a fluoroscopic image showing the location of temperature sensors in the coronary sinus and right ventricular apex of a male 22 Kg mongrel dog using a percutaneous minimally invasive approach for placement in an acute experiment. The location of the occlusive angioplasty balloon in the left anterior descending coronary artery is depicted as well.

FIG. 15 is a tracing of temperature in the right ventricular apex of a male 28 Kg mongrel dog during an acute ischemia experiment in which ischemia was induced by inflation of an occlusive angioplasty balloon in mid portion of left anterior descending coronary artery for the duration of one and two minutes with two minutes of recovery in between.

FIG. 16 is a tracing of temperature in the right ventricular apex of a male 28 Kg mongrel dog during an acute ischemia experiment, in which ischemia was induced by inflation of an occlusive angioplasty balloon in mid portion of left anterior descending coronary artery for the duration of five minutes.

FIG. 17 is a tracing of temperature in the right ventricular apex of a male 28 Kg mongrel dog during an acute ischemia experiment, in which ischemia was induced by inflation of an occlusive angioplasty balloon in distal portion of left anterior descending coronary artery for the duration of one and two minutes with two minutes of recovery in between.

FIG. 19 is a tracing of temperature in the right ventricular apex of a male 28 Kg mongrel dog during an acute ischemia experiment, in which ischemia was induced by inflation of an occlusive angioplasty balloon in distal portion of left anterior descending coronary artery for the duration of five minutes.

FIG. 19 is a tracing of temperature in coronary sinus of an eighty year old male undergoing elective percutaneous balloon angioplasty of an atherosclerotic lesion in the saphenous vein graft to his left anterior descending artery, with a telescoped inlet illustrating the time an occlusive emboli protection device was inflated distal to the lesion.

FIG. 20 is a tracing of temperature in coronary sinus of a seventy-three year old male undergoing elective percutaneous balloon angioplasty of an atherosclerotic lesion in the proximal portion of his native right coronary artery, with a telescoped inlet illustrating the time an occlusive angioplasty balloon was inflated for the first time.

FIG. 21 is a tracing of temperature in coronary sinus of same patient as in FIG. 21, with a telescoped inlet illustrating the time an occlusive angioplasty balloon was inflated for the second time, during the course of which the electrocardiogram showed ST segment elevation.

FIG. 22 is a tracing of temperature and pressure in coronary sinus and pressure in the left main coronary artery of same patient as in FIG. 21. This patient had normal sinus heart rhythm. The telescoped inlet illustrates a typical curve of change in coronary artery flow in a cardiac cycle.

FIG. 23 is a tracing of temperature and pressure in coronary sinus and pressure in the left main coronary artery of a seventy-eight year old male undergoing elective percutaneous balloon angioplasty. This patient had 1^{st} degree block cardiac electrophysiological abnormality.

FIG. 24 is a simultaneous tracing of intramyocardial pressure and temperature recorded in right ventricular apex in a dog in ventricular fibrillation just prior to euthanasia.

### DETAILED DESCRIPTION OF PREFERRED EMBODIMENTS

I. Background: Anatomical Features of the Heart

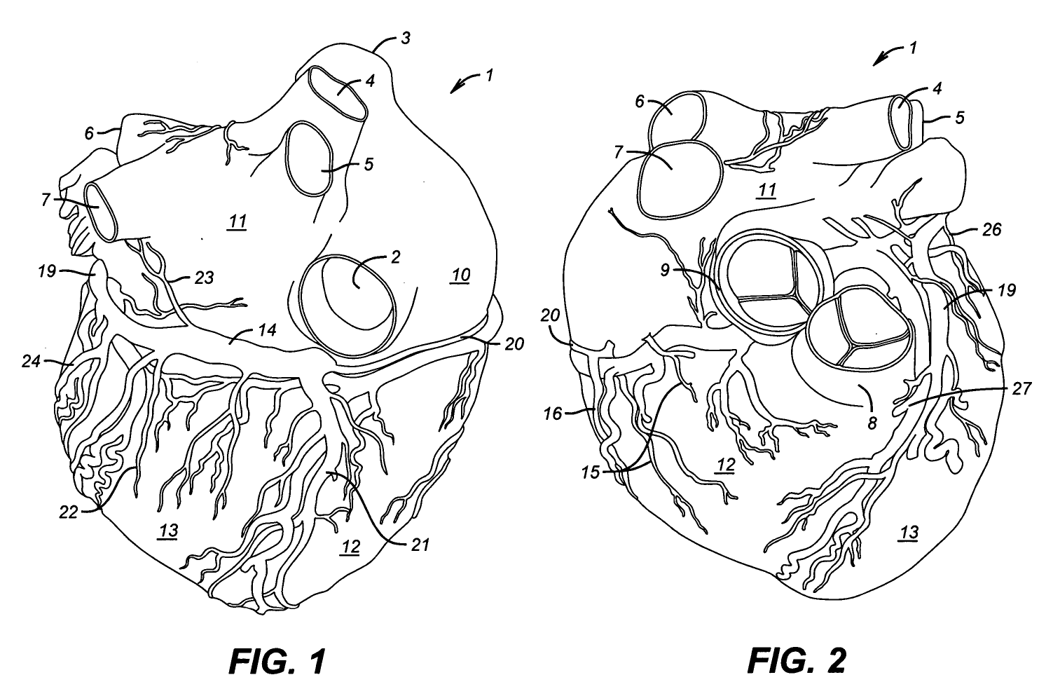

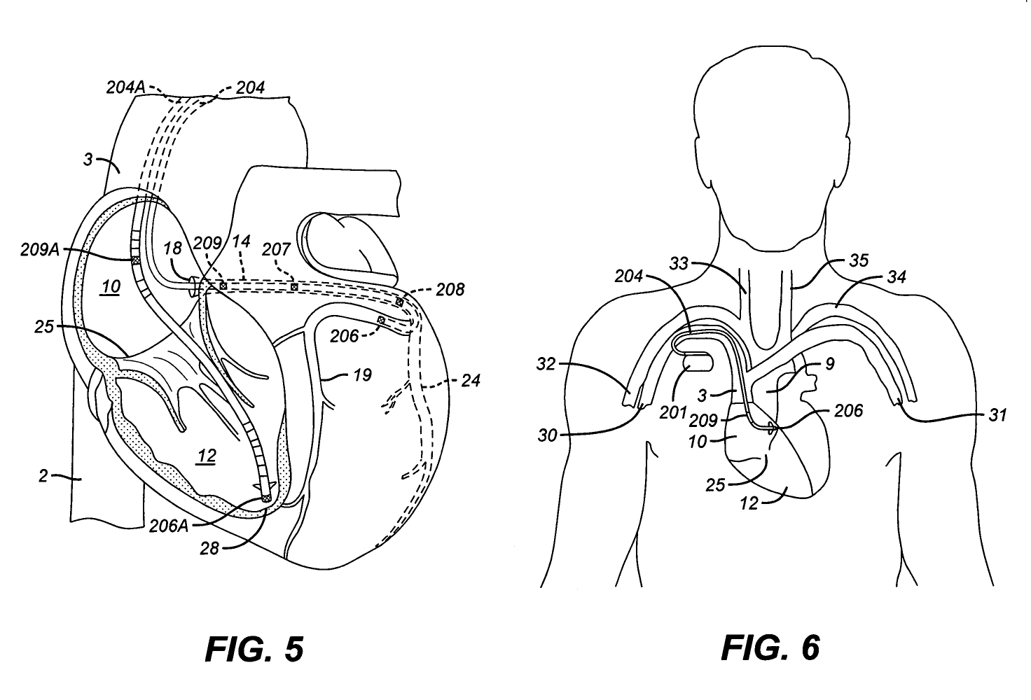

In a specific design of this invention, two or more temperature sensors are located along the coronary sinus lead spread in certain distances so that the gradients among these sensors are determined and such gradients are related to the specific region of the heart that is being drained. To facilitate the description of placement reference is made to FIGS. 1-3 for mention of anatomical features of the human heart 1 and to FIGS. 5-8 for venous access to the coronary sinus. FIG. 1 is a posterior inferior view and FIG. 2 is an anterior superior view, and reference numeral 2 indicates the inferior vena cava, reference numeral 3 indicates the superior vena cava, reference numerals 4 and 5 respectively indicate the superior and inferior right pulmonary veins, 6 and 7 respectively indicate the superior and inferior left pulmonary veins, 8 indicates the pulmonary artery (trunk) and 9 indicates the aorta (trunk). Reference numeral 10 indicates the right atrium, reference numeral 11 indicates the left atrium, reference numeral 12 indicates the right ventricle and reference numeral 13 indicates the left ventricle.

Veins draining the heart may be grouped as: (1) the coronary sinus 14 and tributaries, returning blood to the right atrium 10 from the whole heart (including its septa) except the anterior region of the right ventricle 12 and small, variable parts of both atria and left ventricle 13; (2) the anterior cardiac veins 15 draining an anterior part of the right ventricle 12 and a region around the right cardiac border where the right marginal vein 16 joins this group, ending principally in the right atrium, (3) the venae cordis minimae (Thebesian veins) (not seen), opening into the right atrium and ventricle and, to a lesser extent, the left atrium and sometimes left ventricle.

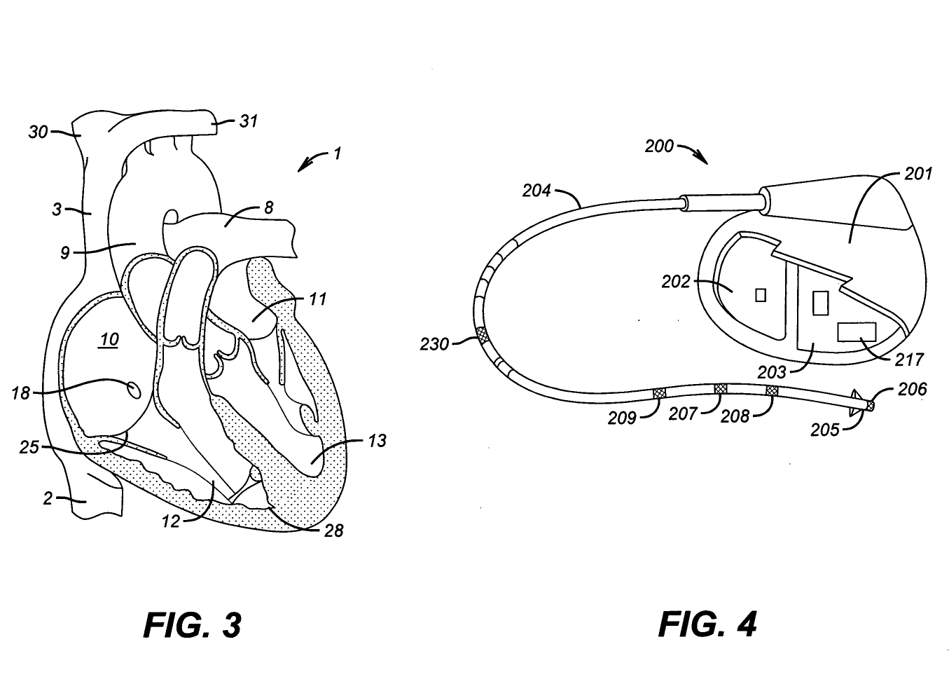

Coronary Sinus: Most cardiac veins drain to the wide coronary sinus 14, about 2 or 3 cm long, lying posterior in the coronary sulcus (atrioventricular groove) between the left atrium 11 and ventricle 13. It ends in the right atrium 10 between the opening of the inferior vena cava 2 and the right atrioventricular orifice 25 (FIG. 3), its opening or ostium 18 (FIG. 3) displaying a semilunar valve (not shown) of the coronary sinus 14. Its tributaries are the great 19, small 20 and middle 21 cardiac veins, and the posterior vein 22 of the left ventricle 13 and the oblique vein 23 of the left atrium 11, all except the last having valves at their orifices. The great cardiac vein 19 enters the beginning of the coronary sinus 14, and receives tributaries from left atrium 11 and both ventricles 12, 13, including the large left marginal vein 24. The small cardiac vein 20 opens into the coronary sinus 14 near its atrial end and receives blood from the back of the right atrium 10 and ventricle (the right marginal vein 16 may join the small cardiac vein 20 in the coronary sulcus (as shown) but more often opens directly into the right atrium 10). The middle cardiac vein 21 opens in the coronary sinus 14 near its atrial end. The posterior vein of the left ventricle 22 usually opens into the center of the coronary sinus 14 but sometimes into the great cardiac vein 20. The oblique vein of the left atrium 22, a small vessel, joins the coronary sinus 14 near its left extremity Anterior Cardiac Veins: The anterior cardiac veins 15 drain the anterior part of the right ventricle 12. The right marginal vein 16 drains adjacent parts of the right ventricle 12, and usually opens separately into the right atrium 11 but may join the anterior cardiac veins 15 or, less often, the coronary sinus 14.

Venae Cordis Minimae: open into all cardiac cavities, and their numbers and size are highly variable.

Cardiac Venous Anastomoses: Most investigators accept widespread anastomosis at all levels of cardiac venous circulation, on a scale exceeding that of the arteries. Not only are adjacent veins often connected but connections also exist between tributaries of the coronary sinus and those of the anterior cardiac veins. Regions of abundant anastomoses are the apex and its anterior and posterior aspects. Like coronary arteries cardiac veins connect with extracardiac vessels, chiefly the vasa vasorum of the large vessels continuous with the heart.

Variation in Cardiac Veins: Attempts to categorize variations in cardiac venous circulation into ‘types’ have not produced any accepted pattern. Major variations concern the general directions of drainage. The coronary sinus 14 may receive all cardiac veins (except the venae minimae), including the anterior cardiac veins 15 (33%), which may be reduced by diversion of some into the small cardiac vein 20 and then to the coronary sinus 14 (28 %); the remainder (39 %) represent the “normal” pattern, as described above. Two major variants based on the course of small cardiac vein 20 have been recognized: a majority (70%) in which the small cardiac vein 20 is independent, small or absent and a less frequent pattern (30%) in which this vein, though variable in size, connects with both coronary and anterior cardiac “systems.”

In a specific design of this invention, two or more of temperature sensors are located along the catheter in the coronary sinus 14 starting with one close to the coronary ostium (OS) 18. Theoccurence of a temperature gradient indicative of ischemia in a subset of these sensors will correspond to the origin of a certain vein draining to the coronary sinus 14, thereby determining the area in which ischemia has occurred, or been more functionally pronounced.

Referring to FIGS. 6-8, access to the coronary sinus is depicted. The anatomical structures indicated in diagrammatic view, in addition to those indicated by reference numeral already designated (superior vena cava 3, right atrium 10, right atrioventricular orifice 25, right ventricle 12 and aorta 9 of heart 1) are the right subclavian vein 30 and left subdlavian vein 31 which empty into the superior vena cava 3. Branching off from aorta 9 in the right side are right subdlavian artery 32 and righr carotid artery 33, and on the left side are left subclavian artery 34 and left carotid artery 35.

II. Summary of the Results of Experiments

A. Animal Experimentation.

Experimentation was done under a protocol approved by the Animal Welfare Committee of the University of Texas Health Science Center in Houston. An acute closed-chest canine model of ischemia was preferred because this setup made it possible to successfully achieve all of the following: i) placement of both CS and RV-Apex sensors; ii) recording electrograms as well as electrocardiogram; iii) induction of myocardial ischemia in different regions of heart; iv) induction of ischemia with different durations; v) progression of ischemia to infarction; vi) CS blood sampling; vii) controlled environment; viii) pathologic necropsy studies including perfusion staining; ix) correlation of electrical and thermometric findings with chemical findings including nitric oxide levels, pH, pO₂, pCO₂, HCO3, base excess (BE), oxygen content and extraction, etc.; x) correlation of electrical and thermometric findings with pathologic finding including area at risk of ischemia and amount of infarction.

In the following discussion of animal studies, reference numerals included within parentheses are the same as the corresponding numbers applied to the anatomical representations of the human heart detailed above.

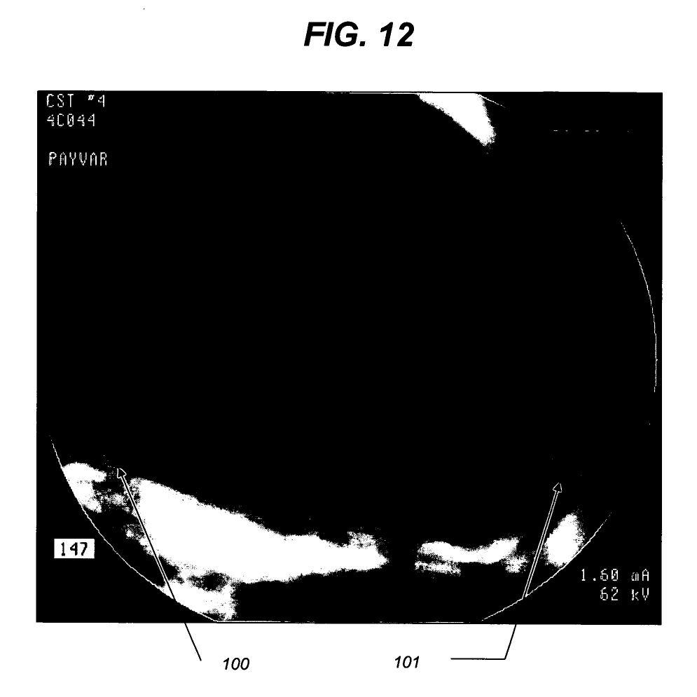

In the very first series of experiments, a temperature sensor was placed through a left lateral thoractomy incision. FIG. 12 shows the fluoroscopic image illustrating the location of temperature sensors in this surgical extra-luminal approach. Reference numeral 100 points to the location of a temperature sensor in coronary sinus (14); number 101 points to location of a temperature sensor in the right ventricular apex (28).

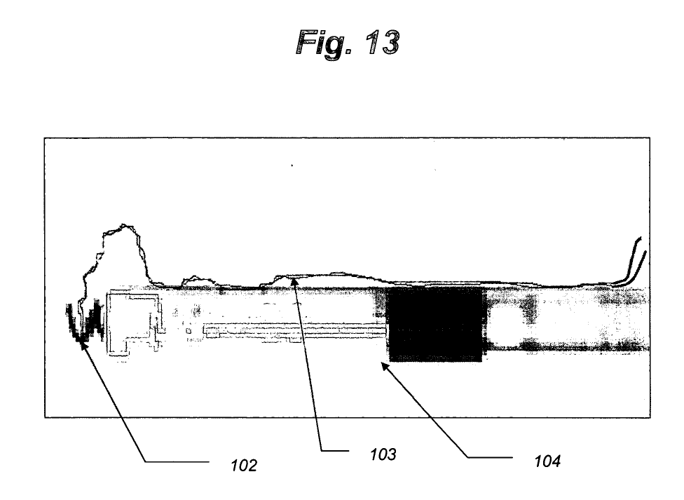

The second phase pilot studies used a percutaneous intra-luminal approach to position a temperature sensing lead that is illustrated in the photographic enlargement which is FIG. 13, in which reference numeral 104 points to an active fixation screw-in active fixation pacing lead, 102 points to a temperature sensor glued on the end of the first turn of the screw; and 103 points to a wire connected to the temperature sensor 102 for signal transmission.

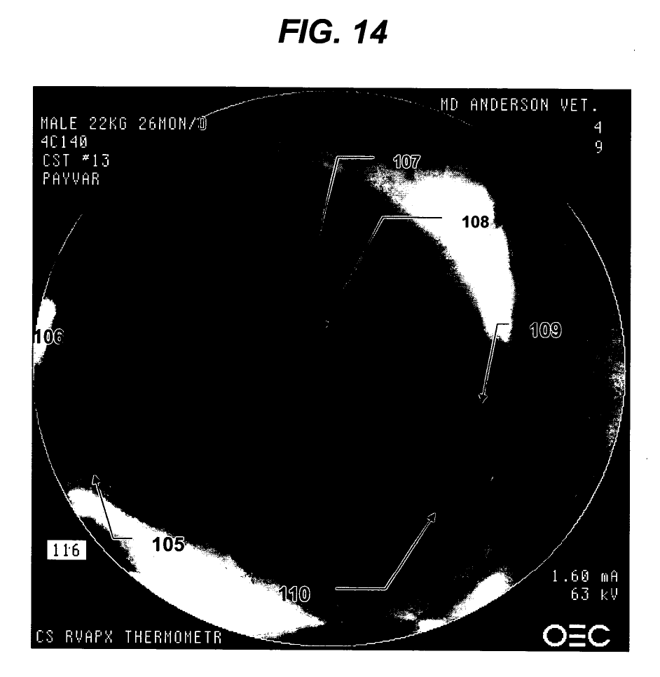

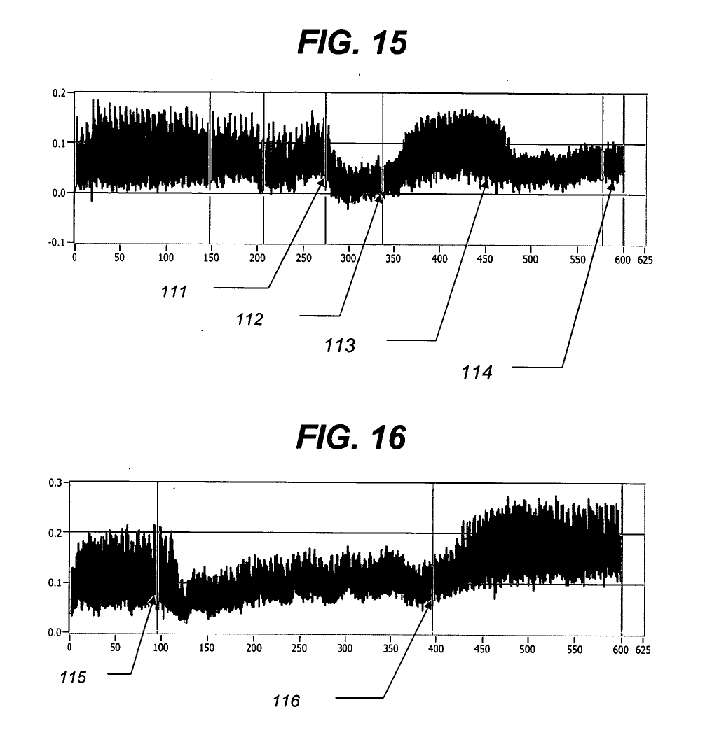

Using a percutaneous intra-luminal approach, myocardial ischemia was induced by inflation of angioplasty balloon 109 in the left anterior decending (LAD) coronary artery (27), in the mid portion (FIG. 14) and distal portion and for different lengths of 1 minute, 2 minutes, and 5 minutes (FIGS. 15-18).

FIG. 14 shows the fluoroscopic image illustrating the location of temperature sensors of the active fixation screw-in active fixation pacing lead 104 in a percutaneous intra-luminal approach. Referring to FIG. 14, reference numeral 105 indicates a catheter in coronary sinus (14); reference numeral 106 indicates the location of a temperature sensor in the coronary sinus (14); reference numeral 107 indicates the floppy tip of the wire in the great cardiac vein (19); reference numeral 108 indicates a guide wire in the left anterior descending (LAD) coronary artery (27); number 109 points to an inflated angioplasty balloon in the distal portion of left anterior descending coronary artery (27); and number 110 points to the location of temperature sensor 102 and the active fixation screw-in bipolar active fixation pacing lead 104 on the screw of which temperature sensor 102 is glued.

Ischemia produced by inflation of the angioplasty balloon 109 was associated with significant decrease in RV apex endomyocardial temperature (FIGS. 15-18). Referring to FIG. 15, the right ventricle apex myocardium (28) temperature sensed by temperature sensor 102 (see FIG. 14) for one and two minute occlusive angioplasty balloon 109 inflations in mid LAD (27) is shown. Reference numeral 111 (FIG. 15) points to a first occlusive angioplasty balloon 109 inflation in mid LAD (27) coronary artery and 112 points to balloon 109 deflation in mid LAD, with a one minute ischemia being induced by inflation of balloon 109 in mid portion of LAD resulting in a significant reduction in temperature of the right ventricle apex endomyocardium between 111 and 112. Reference number 113 points to a second occlusive angioplasty ballon 109 inflation in mid LAD (27) coronary artery and 114 points to balloon 109 deflation in mid LAD, with a two minute ischemia being induced by inflation of balloon 109 in mid portion of LAD resulting in a significant reduction in temperature of the right ventricle apex endomyocardium between 113 and 114.

Referring to FIG. 16, the right ventricle apex myocardium (28) temperature sensed by temperature sensor 102 for a five minute occlusive angioplasty balloon 109 inflations in mid LAD (27) is shown. Reference numeral 115 (FIG. 16) points to a first occlusive angioplasty balloon 109 inflation in mid LAD (27) coronary artery and 116 points to balloon 109 deflation in mid LAD, with a five minute ischemia being induced by inflation of balloon 109 in mid portion of LAD resulting in a significant reduction in temperature of the right ventricle apex endomyocardium between 115 and 116.

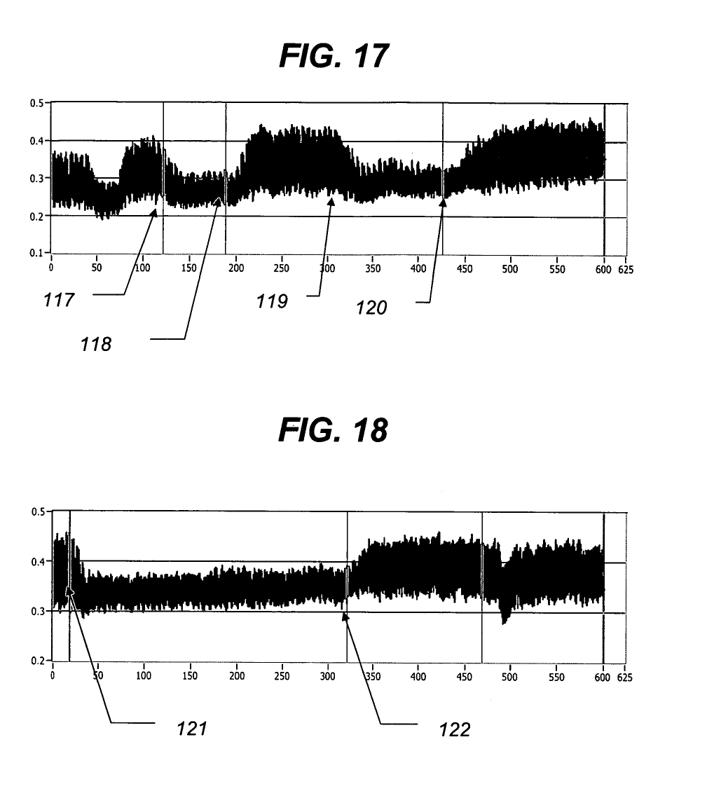

Referring to FIG. 17, the right ventricle apex myocardium (28) temperature sensed by temperature sensor 102 for one and two minute occlusive angioplasty balloon 109 inflations in distal LAD (27) is shown. Reference numeral 117 (FIG. 15) points to a first occlusive angioplasty balloon 109 inflation in distal LAD (27) coronary artery and 118 points to balloon 109 deflation in distal LAD, with a one minute ischemia being induced by inflation of balloon 109 in the distal portion of LAD resulting in a significant reduction in temperature of the right ventricle apex endomyocardium between 117 and 118. Reference number 119 points to a second occlusive angioplasty ballon 109 inflation in distal LAD (27) coronary artery and 120 points to balloon 109 deflation in distal LAD, with a two minute ischemia being induced by inflation of balloon 109 in distal portion of LAD resulting in a significant reduction in temperature of the right ventricle apex endomyocardium between 119 and 120.

Referring to FIG. 18, the right ventricle apex myocardium (28) temperature sensed by temperature sensor 102 for a five minute occlusive angioplasty balloon 109 inflations in distal LAD (27) is shown. Reference numeral 121 (FIG. 16) points to a first occlusive angioplasty balloon 109 inflation in distal LAD (27) coronary artery and 122 points to balloon 109 deflation in distal LAD, with a five minute ischemia being induced by inflation of balloon 109 in distal portion of LAD resulting in a significant reduction in temperature of the right ventricle apex endomyocardium between 121 and 122.

We used Fourier transformation, cosinor analyses and linear mixed models to study the numerical properties of the changes in right ventricle apex temperature (T-apex) sensor in association with ischemia. The graphical illustration of the nature of these changes is presented in FIGS. 15-18. One minute of ischemia caused 0.03° C. fall in average of T-apex (P<0.0001). Temperature had an overshoot of 0.01° C. following release of ischemia (P<0.0001), although the resolution of the temperature sensing device was not enough to establish this relation. Subsequent episodes of ischemia caused smaller change in the average of T-apex, probably because of the conditioning process that took place during repeated episodes of experimentally induced fibrillation, indicating continued similarity of these two waves throughout this terminal rhythm abnormality ischemia. The frequency of the T-apex matched that of heart beat. It increased by 1-3 beats/minute during ischemia, and returned to pre-ischemic values after the release of occlusion. The effect of ischemia on the amplitude of T-apex signal waveform is noteworthy. Prior to ischemia, the amplitude of the main harmonic of the underlying Fourier transform of the wave was 0.018° C. During ischemia it fell down to 0.009° C., and after release of ischemia it went up to 0.029° C. Then again it is of note that the resolution of the temperature sensing device was not enough to establish these relations. In cases where the heart went into episodes of irregular rhythm, the waveform generated by the temperature sensor stayed true to the waveform of local pressure, indicating continued local heat production.

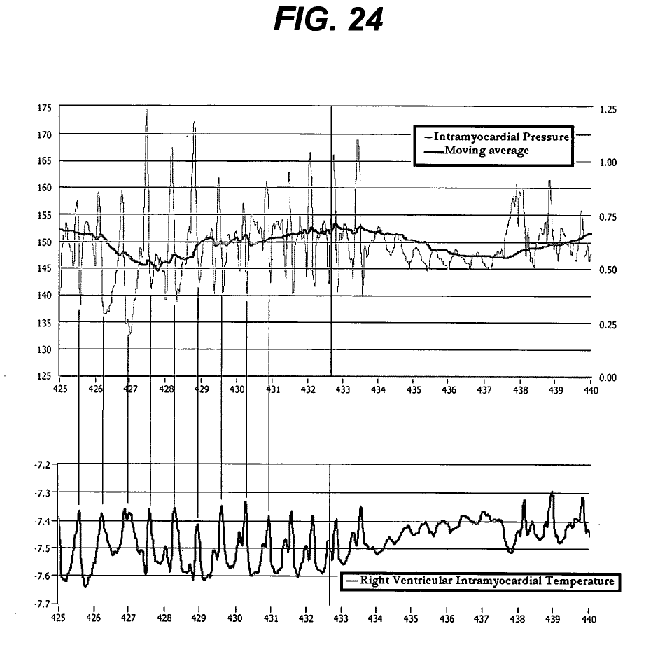

Reference is made to FIG. 24, in which the horizontal axes are time in seconds; the bottom left vertical axis is temperature (° C.); the top left vertical axis is intramyocardial pressure at right ventricular apex (mmHg); the bottom curve is intramyocardial temperature at right ventricular apex relative to intramyocardial temperature at baseline; the top curves are actual value (gray line) and moving average (dark line) of intramyocardial pressure at right ventricular apex. Midline vertical line indicates the time euthanasia was performed. FIG. 24 illustrates simultaneous tracing of local right ventricle apex temperature and pressure in a dog during ventricular fibrillation, indicating continued similarity of these two waves throughout this terminal rhythm abnormality.

B. Human Experimentation.

Eight patients undergoing elective coronary angioplasty and stenting were enrolled in the study. The study was approved by the hospital's institutional review board. Patients were excluded if they had history of recent myocardial infarction, contraindications for coronary sinus catheterization, or fever. Coronary sinus temperature (T_{CS}) measurement started before the angioplasty catheter was introduced, and continued until after the left-sided intervention was completed. The course of angioplasty and stenting was unaltered. T_{CS} was continuously measured using RADI PressureWire4 (RADI Medical Inc., Reading, Mass., USA.) The sensor was placed in the coronary sinus (CS) (reference numeral 14) at the junction of CS with great cardiac vein (19). Temperature measurement was done at a rate of 100 samples per second with sensitivity of ±0.05° C.

Statistical analysis was done using SAS software version 8.2 (SAS Institute, Cary, N.C., USA). Time series graph smoothing was done using Reinsch's cubic spline functions method. Simultaneous measurements of pressure in the coronary sinus were included in all the graphs, using the same smoothing method. The temperature difference between right atrium (T_{RA}) and T_{CS}, the effect of ischemia on T_{CS} (T_{CS} during balloon inflation) and the effect of removal of atherosclerotic lesion on T_{CS} (the difference of T_{CS} before balloon inflation and after balloon deflation) were estimated using linear mixed effects regression. Influence and outliers were studied using ordinary least squares regression on individual patient tracing, with no intention of using the study of outliers and influence to correct the estimators of the mixed effects model.

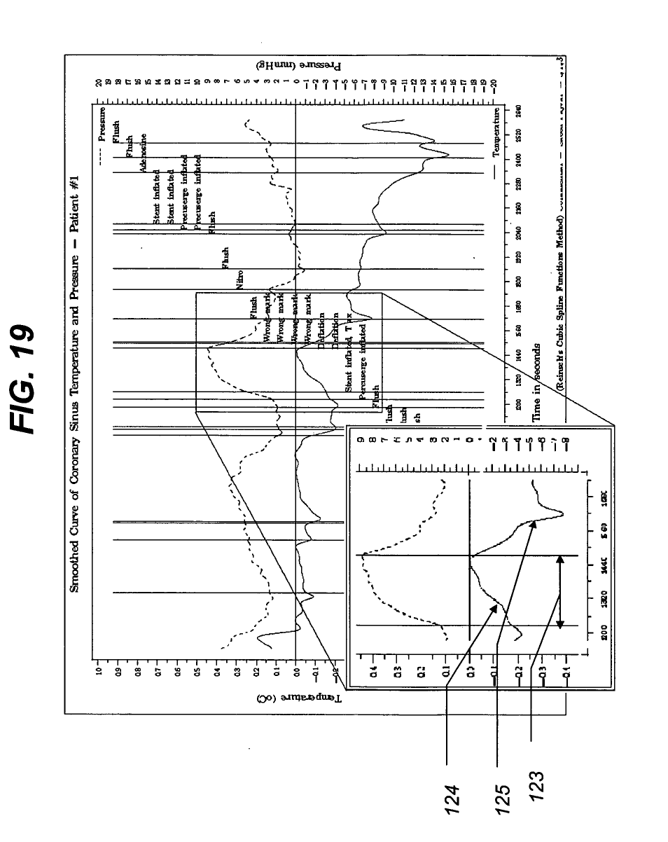

All patients were male. Their ages had a mean of 68 years ±10 (SD). Seven were Caucasian and one was African American. Five had presented with Unstable Angina and the remaining had presented with Stable Angina. Three of the target lesions were native and the remaining were saphenous vein grafts. Coronary sinus catheterizations were uneventful. Simultaneous tracing of coronary sinus pressure and temperature during the whole procedure in a typical case is illustrated in FIG. 19, in which the horizontal axis is time in seconds; the left vertical axis is temperature (° C.); the right vertical axis is pressure (mmHg); the continuous line is blood temperature in coronary sinus relative to blood temperature in the right atrium at baseline (“baseline” in these and the subsequent figures in this human study means the temperature of the right atrium at the time of insertion of the sensor into the heart, the place where the baseline is established); the dashed line is pressure in the coronary sinus. Reference numeral 123 indicates five minute ischemia induced by inflation of emboli protection occlusive balloon distal to atherosclerotic lesion in saphenous vein graft to left anterior descending coronary artery. FIG. 19 shows a steep rise 124 in temperature and pressure was seen immediately after balloon inflation, and a fall 125 below baseline was observed after balloon deflation.

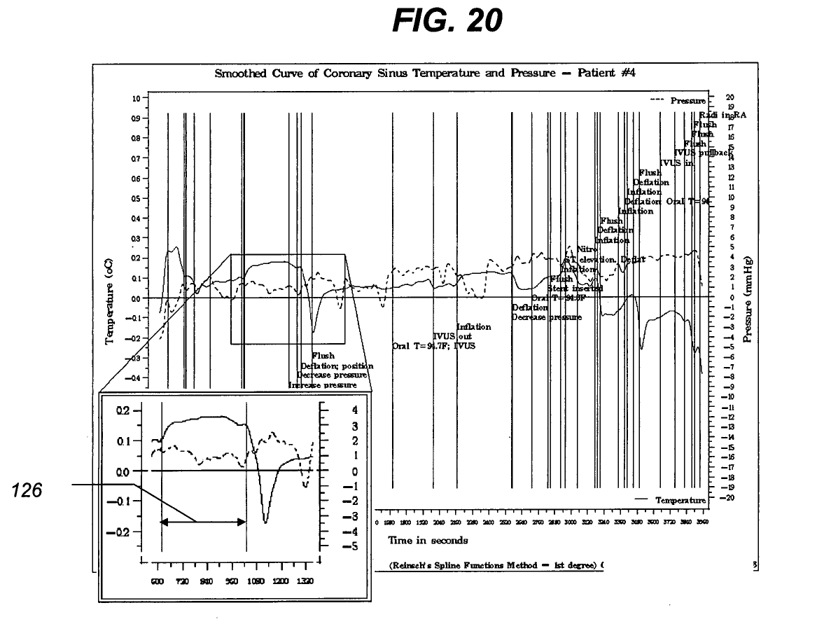

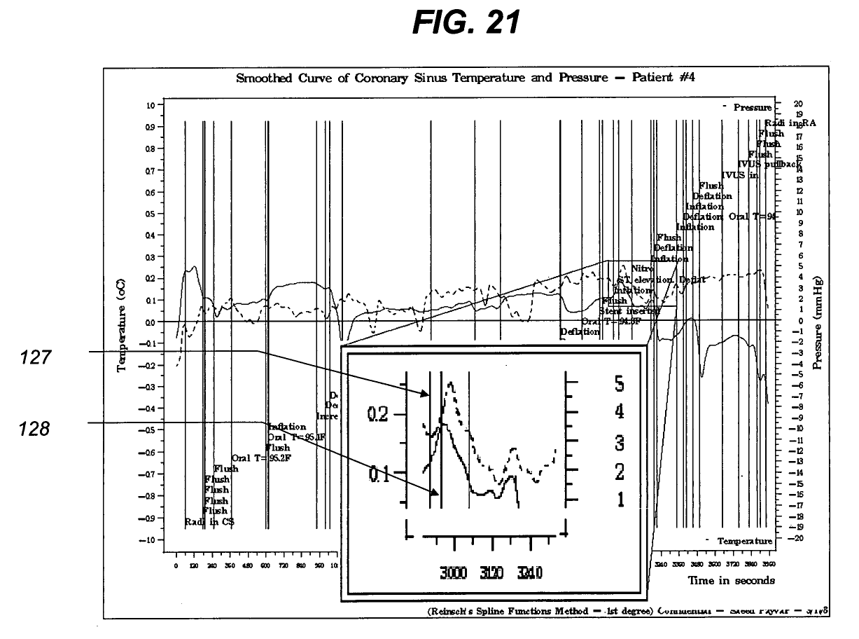

Referring to FIG. 20, the axes and trace lines are as in FIG. 19. Reference numeral 126 in FIG. 20 indicates a one minute ischemia induced by inflation of angioplasty occlusive balloon over an atherosclerotic lesion in mid portion of native right coronary artery. The results were a pattern in a similar fashion to the one seen in FIG. 19, but it was not observed in one patient who had significant collateral circulation. Three patients experienced chest pain during the procedure. One of these patients had ST elevation. The trace of the patient having ST elevation is shown in FIG. 21, in which the axes and trace lines are as in FIGS. 19 and 20. Referring to FIG. 21, reference numeral 127 indicates start of two minute ischemia induced by inflation of angioplasty occlusive balloon over an atherosclerotic lesion in mid portion of native right coronary artery, and reference numeral 128 indicates occurrence of ST segment elevation. The temperature rise preceded pain or ST-elevation in these patients.

Following inflation of the balloon, T_{CS} significantly increased (mean adjusted increase of 0.20° C., p<0.0001). Upon restoration of coronary flow with the deployment of the stent, T_{CS} deceased below the baseline (mean adjusted decrease of 0.83° C., p<0.0001). We concluded that:
1) A significant increase in T_{CS} was associated with ischemia.
2) This rise preceded electrocardiographic changes and development of symptoms.
3) A significant fall in T_{CS} below baseline was associated with the removal of ischemia.
   1. Coronary Sinus
   a. The Discovery of Signal Waveform of Temperature Sensor Located In Coronary Sinus.

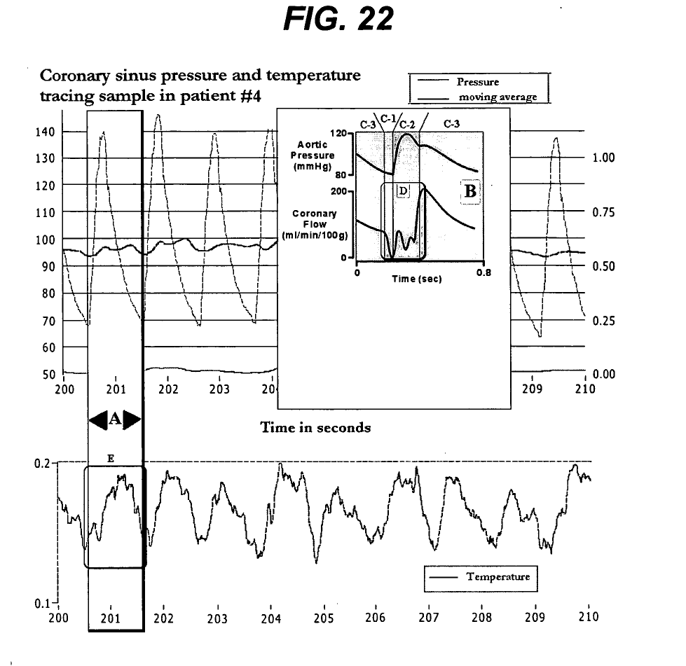

Reference is made to FIG. 22, in which the horizontal axes is time in seconds; the bottom left vertical axis is temperature (° C.); the top left vertical axis is pressure (mmHg); the bottom curve is blood temperature in coronary sinus relative to blood temperature in right atrium at baseline; the top curves are actual value (gray line) and moving average (dark line) of pressure in left main coronary artery. Box A demarks a cardiac cycle; box C is an illustration showing curve of typical change in coronary artery flow in a cardiac cycle; C-1 is isovolemic contraction phase of systole in a cardiac cycle; C-2 is ejection phase of systole; C-3 is diastole; box E is the curve of signal from temperature sensor resembling that of the coronary flow. As depicted in FIG. 22, the signal that was received from the temperature sensor had a waveform. The patient in whom the tracing of FIG. 22 was recorded had a regular (sinus) heart rhythm, and the waveform of the signal generated by the temperature sensor closely traced cardiac cycle. It ran with a phase in parallel with changes of blood pressure in cardiac left main coronary artery. This waveform resembled that of coronary blood flow, indicting that this waveform might have been produced by flow of blood around the temperature sensor.

We speculate that these changes indicated transfer of thermal energy through convection and by a rapidly moving vehicle (flowing blood), as opposed to most other biological temperature measurements where heat transfer is through either of diffusion or convection via slow moving vehicles. In our application in which temperature of blood in coronary sinus is being measured, because the flow of blood in coronary sinus is pulsatile in nature, measuring temperature continuously and with high sampling rate proved helpful in discovering the waveform of the signal generated by the temperature sensor. Traditionally, temperature measurements in most biologic environments have been considered low frequency signals, i.e. slowly changing. However, those measurements indicated transfer of thermal energy through either diffusion, which is comparatively slower, or convection by a slow moving vehicle, such as extra-cellular fluid. In our application, on. the other hand, temperature measurements showed a rapidly changing oscillating form that paralleled pressure and flow waveforms, indicating that the underlying process was not diffusion or a slow convection process.

It will be observed that we have referred to the signal waveform of the temperature sensor in the coronary sinus, not the temperature of the cardiac sinus. The use of the term “signal waveform” requires further explanation. The coronary sinus is a tube that maintains low pressure. It expands with pulsatile flow of blood dramatically more as compared to arteries, and even at times collapses. The temperature sensor in the coronary sinus measures two types of temperature, one is that of blood that passes through the coronary sinus, and the other is the temperature of the coronary sinus wall when coronary sinus collapses, as during systole when flow almost stops because of an increase in myocardial pressure surrounding coronary sinus, or because of an increase in pressure in the thoracic cage, as during exhalation. If collapse happens, a temperature sensor in the coronary sinus will measure the temperature of the coronary sinus wall, which is more reflective of myocardial temperature in that location. Therefore, it is the signal generated by the sensor in the cardiac sinus that has a waveform. By an approach focusing on waveform characteristics, one has the advantage of analyzing the characteristics of a wave, which in turn provides more information through the analysis of its shape and its relation to other biologically important waves.

A brief definition of terms used in general description of a waveform is given here for clarity purposes. We use the term “average” to describe the mean of the oscillation. When talking about waveforms, an average is the value around which the signal oscillates; the term is equivalent to “MESOR,” an acronym for midline estimating statistic of rhythm, which is used in literature pertaining to cosinor analysis of biological rhythms. The “amplitude” is the distance between the maximum and minimum of oscillation is a certain cycle. This is analogous to ‘beat to beat’ variation of temperature measured in a certain location. The “period” is the time of a complete cycle of oscillation. The “frequency” is the number of a complete cycle of oscillation in unit time. The “phase” is the timing of the cosine maximum, and is an equivalent of acrophase used in cosinor analysis literature. Cosinor analysis is common among those who work on biologic rhythmometery. A good review is Nelson W, Tong Y L, Lee J K, Halberg F. Methods for cosinor-rhythmometry. Chronobiologia. 1979 October-December;6(4):305-23. (Using sea waves as an example, the time distance between two prominent crests reaching the shore is the phase between them.)

Based on our human experimentation, we have discovered that:
1) The waveform of the signal generated by a temperature sensor located in coronary sinus (T_{CS} signal waveform) traced that of the pressure in coronary artery with a phase.
2) The shape of T_{CS} signal waveform in normal heart rhythm resembled that of typical coronary blood flow with a phase.
   b. The Discovery of the Effect of Myocardial Ischemia on Signal Waveform of Temperature Sensor Located In Coronary Sinus.

As explained above, and as shown in relation (4), temperature of coronary sinus blood is directly related to the heat production of the heart and inversely related to the amount of flow to the heart. Ischemia is caused by a decrease in flow, and in itself causes dyskinesia, which in turn may increase heat production (inefficiency). We discovered that:
1) The frequency of T_{CS} signal waveform increased during ischemia, indicating reactive tachycardia caused by ischemia.
2) The amount of increase of frequency of T_{CS} signal waveform during ischemia was proportional to size of ischemic region.
3) The average of T_{CS} signal waveform increased during ischemia, indicating decrease in overall coronary flow and increase in myocardial heat production because of reactive hyperkinesias or dyskinesia of non-ischemic areas of heart.
4) The amount increase of average of T_{CS} signal waveform was proportional to size of ischemic region.
5) The slope of increase (rate of increase) of average of T_{CS} signal waveform during ischemia was rapid at first, consequently becoming slow and reaching plateau. This indicated two possible mechanisms behind the process, i) an abrupt and rapid decrease in flow causing an abrupt and rapid increase in temperature, followed by ii) hyperkinesias or dyskinesia of non-ischemic areas of the heart, causing increased heat production (cardiac inefficiency) and a slow increase of temperature of coronary sinus blood.
6) The amplitude of T_{CS} signal waveform decreased during ischemia, indicating decrease in overall coronary flow.
7) The amount of decrease of amplitude of T_{CS} signal waveform was proportional to size of ischemic region.
   b. The Discovery of the Effect of Release of Myocardial Ischemia on Signal Waveform of Temperature Sensor Located In Coronary Sinus.

Following release of ischemia, there is an abrupt increase in coronary flow, which is indicative of neurohormonal as well as local regulatory mechanisms that become active during ischemia. We discovered that:
1) The frequency of T_{CS} signal waveform decreased after release of ischemia, down to pre-ischemic values.
2) The average of T_{CS} signal waveform decreased after release of ischemia, to values below pre-ischemic values, indicating reactive hyperemia.
3) The slope of the decrease of average of T_{CS} signal waveform following release of ischemia was sharper than the slope of increase of same following ischemia, indicating a flow dependent fall of temperature caused by two mechanisms, removal of ischemia and reactive hyperemia.
   c. The Discovery of Signal Waveform of Temperature Sensor Change with Rhythm Abnormality

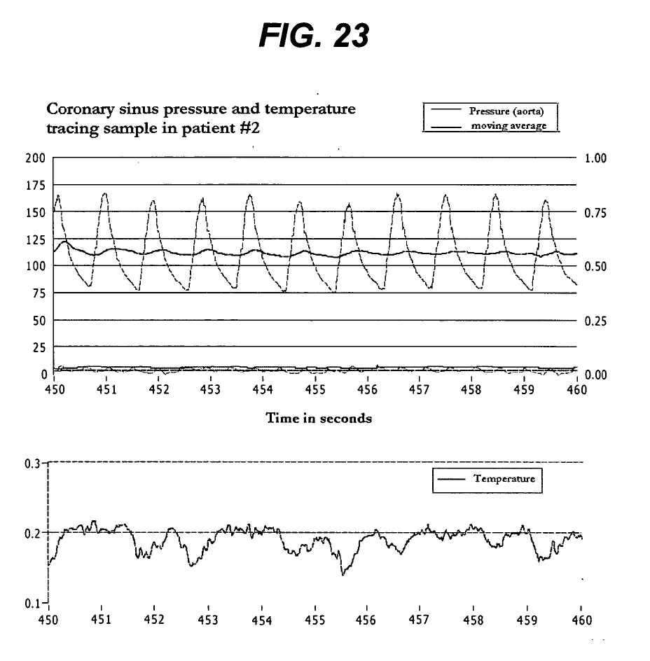

It is of note that the relation of T_{CS} signal waveform and coronary flow did not stay the same in situations in which there was a rhythm abnormality. Reference is made to FIG. 23, in which the horizontal axes are time in seconds; the bottom left vertical axis is temperature (° C.); top left vertical axis is pressure (mmHg); the bottom curve is blood temperature in coronary sinus relative to blood temperature in right atrium at baseline; the top curves, actual value (gray line) and moving average (dark line) of pressure in left main coronary artery. FIG. 23 illustrates the simultaneous tracing of temperature and pressure in coronary sinus and pressure in the left main coronary artery of a seventy-eight year old male undergoing elective percutaneous balloon angioplasty who had 1st degree block cardiac electrophysiological abnormality. As illustrated in this tracing, although T_{CS} still illustrate a period undulation behavior, the form is different in character from the typical shape of coronary flow, opening the possibility of detecting heart rhythm abnormalities in the course of the study of T_{CS} wave form. The combination of T_{CS} wave form with electrocardiogram, or electrogram, is helpful in determining how an electrical abnormality translates into changes in coronary flow and myocardial inefficiency and provides a new tool for studying the relation of rhythm abnormalities and their effect on myocardial efficiency and energetics. This would in turn provides a physiologic criterion, or a set of criteria, for “pacing for survival”, being a means of fine tuning pacing rates that increase survival not only by preventing life threatening arrhythmias but also by preventing myocardial inefficiency and damage by limiting overpacing.

2. Right Ventricular Apex

a. Signal Waveform of Temperature Sensor Located In Right Ventricular Apex.

Based on our experimentation in animals (discussed above), we discovered that:
1) The signal generated by a temperature sensor located in right ventricular apex muscle has a periodic waveform (RVA-T signal waveform).
2) The frequency of RVA-T signal waveform is equal to heart rate.
3) The shape of RVA-T signal waveform in normal heart rhythm resembled that of typical coronary blood flow with a smaller amplitude.

The relatively small undulations of signal generated by a temperature sensor in right ventricular apex myocardium probably originate from the flow of blood through the muscular mass, which is pulsatile in nature, and transfers heat out of this environment.

b. The Discovery of the Effect of Myocardial Ischemia on Signal Waveform of Temperature Sensor Located In Right Ventricular Apex Myocardium.

1) The frequency of RVA-T signal waveform increased during ischemia, indicating reactive tachycardia caused by ischemia.
2) The amount increase of frequency of RVA-T signal waveform during ischemia was proportional to size of ischemic region.
3) The average of RVA-T signal waveform decreased during ischemia, indicating decrease in overall coronary flow and decrease in local myocardial heat production because of ischemia.
4) The amount increase of average of RVA-T signal waveform was proportional to size of ischemic region.
5) The slope of decrease (rate of decrease) of average of RVA-T signal waveform during ischemia was rapid at first, consequently becoming slow and reaching plateau..
6) The amplitude of RVA-T signal waveform decreased during ischemia, indicating decrease in overall coronary flow.
7) The amount decrease of amplitude of RVA-T signal waveform was proportional to size of ischemic region.
   c. The Discovery of the Effect of Release of Myocardial Ischemia on Signal Waveform of Temperature Sensor Located In Right Ventricular Apex Myocardium.
1) The frequency of RVA-T signal waveform decreased after release of ischemia, down to pre-ischemic values.
2) The average of RVA-T signal waveform increased after release of ischemia, to values above pre-ischemic values, indicating reactive hyperkinesias.
3) The slope of the increase of average of RVA-T signal waveform following release of ischemia was sharper than the slope of decrease of same following ischemia.
4) The amplitude of RVA-T signal waveform increased after release of ischemia, to values above pre-ischemia levels, indicating compensatory overshoot of myocardial heat production.

Although the foregoing tests were conducted with a lead attached into the apex of the right ventricle, the invention as respects an apical location of a sensor is not so limited. One or more apical sensors may be located at the apex. For example, a sensor could be located at the left ventricle apex alone, or a pair of spaced sensors set could be used, one sensor being 2 or 3 mm proximate of the other, the more distal being screwed into the left ventricle apex and the more proximal one being screwed into the right ventricle apex. Temperature measurements from the left ventricle sensor would furnish information as to stenosis of the left coronary artery and temperature from the right ventricle apex would furmish information about stenosis of the right coronary artery.

The coronary sinus drains mostly the regions of the heart supplied by the left coronary artery, draining only some of the heart fed by the right coronary artery. Using a combination of thermal measurements from a coronary sensor and an apical sensor, a pattern of increased temperature increases sensed by the coronary sinus while apex temperatures remained constant would indicate a stenosis of the arteries of the left side of the heart. Conversely, T_{CS} remained constant and T-apex decreased, stenosis of the right side of the heart would be indicated. On the other hand, if T_{CS} and T-apex both increase, but if core temperature as for example from the right atrium remained constant, inflammation in the coronary circulation would be indicated. A gradient increase from core to apical temperatures is a sign of coronary artery inflammation, as is a gradient increase from core to coronary sinus temperatures.

III. Physiologic Principles Explaining the Discoveries Concerning Temperature Increase in the Coronary Sinus Following Myocardial Ischemia and Infarction

The following offers a physiological explanation for the discoveries concerning temperature increase in the coronary sinus following myocardial ischemia and infarction.

Myocardial metabolism in left ventricular releases energy in order to perform work on the circulation. The mechanical efficiency of the left ventricle is less than 100%, thus some energy is “wasted” as heat. The energy balance of the left ventricle, under aerobic conditions, can be described by the equation
EE_{O}_{₂}=H_{LV}+Pₑₓₜ (1)
where EE_{O}_{₂} is the energy equivalent of oxygen extracted in unit time, H_{LV} is is the total left ventricular heat production in unit time, and Pₑₓₜ is the external power produced by left ventricle.

A small proportion of the total heat produced (H_{LV}) is used up in the endothermic reactions of oxygen and carbon dioxide with hemoglobin (H_{chem}), but the greater proportion is removed from the myocardium by the coronary circulation, and by diffusion into the mediastinum, and ventricular cavities. This component of the total heat production, the external heat loss (Hₗₒₛₛ), equal to H_{LV} minus H_{chem} may be expressed as
H_{LV}−H_{chem}=Hₑₓₜ=H_{CS}+H_{diff} (2)
where H_{CS} is the heat removed by the coronary circulation, and H_{diff} is the heat loss by diffusion into the mediastinum and ventricular cavities.

This invention includes measurement of blood temperature in coronary sinus. This temperature is a reflection of the amount of heat added to coronary venous blood (H_{CS}). In this invention, for practical and safety purposes, the heat contribution of the myocardium to the coronary circulation is calculated as the gradient of temperature in the coronary sinus (T_{CS}) and the temperature in the right atrium or the core body temperature (T_{C}), the gradient being denoted by T_{CS}−T_{C}. This arrangement is chosen for two reasons. First, continuous measurement of temperature in the right atrium causes much less risk of embolization, and second, the right atrium temperature provides a very stable background measurement. However, it is inferior to temperature measurements made in the root of the aorta in situations in which the temperature of blood that enters the coronary circulation is critically affected after passing through the respiratory system, such as breathing very cold air.

H_{CS} has the following relation with temperature difference between aortic and coronary venous blood, which approximates finely to T_{CS}−T_{C}, coronary sinus flow rate, and the density and specific heat of blood. This relationship is expressed in the equation
H_{CS}=Q_{CS}p_{b}C_{b}T_{diff} (3)
where Q_{CS} is the left ventricular blood flow (ml/min) (which approximate finely to coronary sinus flow), P_{b} is the density of blood (1.36 g/ml), C_{b} is the specific heat of blood (3.6 J/° C.g) (constant), and T_{diff} is the temperature difference between aorta and coronary sinus (° C.). Coronary sinus blood flow is a reliable indicator of total left ventricular blood flow in humans.

In a specific design of this invention, as described below, flow in the coronary sinus is measured. This measurement may be used to determine the extent of change in H_{CS} as shown in (3), giving a better estimate of occurrence and extent of ischemia.

Equation (3) can be rewritten as the following:

$${T}_{CS} \approx c \frac{Heat}{Flow} \qquad ( 4 )$$

As depicted in relation (4), a decrease of flow to a specific region of the heart will lead to an increase in T_{CS}. This is akin to the temperature rise in cooling fluid of an engine when there is a shortage of radiating fluid going through the engine. However, blood plays multiple roles, for decrease of blood flow to myocardium to the extent that it would cause ischemia will lead to immediate changes in the function of the affected area, as well as prompt reactive and compensatory changes in the function of non-ischemic areas. This causes an immediate decrease in heat production in the affected area and a delayed increase in heat production in non-ischemic areas. Therefore, there will be changes in the same direction in both the right hand side and left hand side of equation (3). As depicted in relation (4), these changes would have a net effect toward increase in T_{CS}. Moreover, discordance in the amount of change in flow and heat production causes a temperature change detectable in the coronary sinus.

In addition to that, accompanying ischemia and dysfunction of the ischemic region, there will be initially hyperkinesis of the remaining normal myocardium, the result of acute compensatory mechanisms, including increased activity of the sympathetic nervous system and the Frank-Starling mechanism. A portion of this compensatory hyperkinesis is ineffective work because contraction of the unaffected segments of myocardium causes dyskinesis of the ischemic zone.

Ischemia might be caused by clots, which may dislodge. Ischemia may also be caused by spasm of heart arteries, and these resolve. Studies by Maseri and his colleagues (reference 24) showed that a patient might have even up to 14 episodes of painless ischemia in a day. Following endothelial injury over an atherosclerotic lesion, which can be spontaneous at times, there will be an aggregation of platelets. This platelet aggregate causes reduced flow, and probably ischemia, yet it more than often dislodges, and flow is restored. This process sometimes goes into a self-repeating cycle, called cyclic flow variation. Ischemia can arise form other causes, including atherosclerotic blockage. If an ischemia causes infarction, the ischemia and the infarction are most accurately and earlier detected by signature initial changes in temperature and temperature waveform as described in detail below. If infarction results, the signature changes occurring with ischemia are followed 1-12 hours later by a rise in temperature caused by inflammation first from neutrophils involved in reperfusion injury, depending on the severity of the ischemia and the timing of reperfusion as clot and spasm resolve (spontaneously or due to medications or balloon angioplasty), then beginning at about 12-24 hours and continuing for weeks from macrophages involved in repair. Finally, the normalization of temperature is helpful in determining the completion of healing, at which time it is safe to exercise and start use of steroids. This time also is a good one for evaluating left ventricular function and arrhythmia.

IV. Apparatus

This invention involves detection of ischemia, and infarction, based on the analysis of blood temperature in the venous drainage system of the heart, among a plurality of other physiologic indicators.

A. Sensing and Control System

Referring to FIG. 4, a sensing apparatus 200 includes a housing 201 which includes a battery 202 and a control unit 203 in electrical communication with the battery. Central processing unit 203 is in electrical communication, by wired or wirelsss means, with a distal sensor set 206, differential sensor sets 207, 208 and proximal sensor set 209 along lead 204 and lead tip 205. Optionally the lead 204 includes an electrically connected accelerometer 230. Sensor sets 206-209 contain capabilities to detect one or more of temperature, pressure, flow, pO₂, pCO₂, pH, electrogram, and nitric oxide (NO). As thermal sensor sets, sets 206-209 may be thermisters or other suitable device known in the art for transforming sensed temperatures into electrical signals, and are described as thermal sensors in FIGS. 5-8.

Lead 204 is placeable in the heart, suitably using an over the wire system or stylet or both, in a manner known to those skilled in the art. As shown in FIGS. 6-8, the housing set is implanted in the patient. The lead is moved through the vasculature of the patient as by the right subdlavian vein 30, thence to the superior vena cava 3, thence into the right atrium 10 for disposition thence.

Referring to FIG. 6, use is made of subset of the features of apparatus 200 for placement of one thermal sensor set in the right atrium and another thermal sensor set in the coronary sinus. Lead 204 passes from implanted housing 201 through the right subclavian vein 30, thence to the superior vena cava 3, thence into the right atrium 10 thence through coronary sinus ostium 18 for location in the coronary sinus 14, with two sensor sets, one 209 in the right atrium 10 and one 206 in the coronary sinus 14 close to the coronary sinus ostium 18. These sensors are connected, through wireless or wired means, to control unit 203 in implanted housing 201 that alternatively can be a package worn externally by the patient.

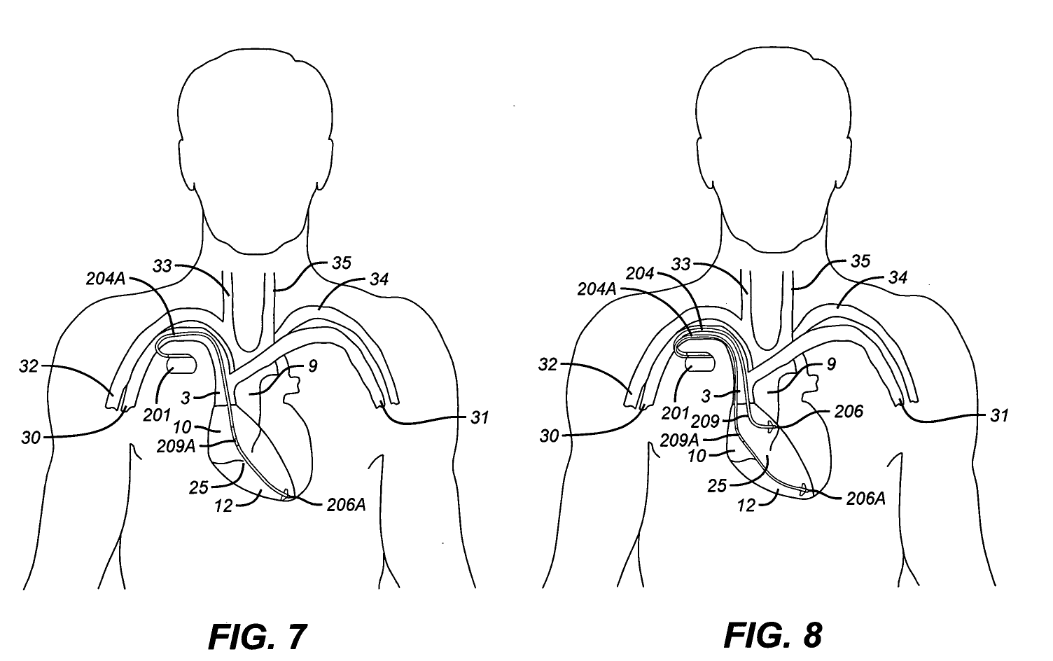

Referring to FIG. 7, use is made of another subset of the features of apparatus 200 for placement of one thermal sensor set in the right atrium 10 and another thermal sensor set in the right ventricle apex 28. Lead 204A passes from implanted housing 201 through the right subdlavian vein 30, thence to the superior vena cava 3, thence into the right atrium 10 thence through the right ventricle 12 to the right ventricular apex 28, with sensor set 209A in the right atrium 10 and sensor set 206A in the right ventricular tip 205A of lead 204A.

Referring to FIG. 8, in another use, both the above said leads are employed.

Referring to FIG. 5, in a preferred use, two leads are used as in FIG. 8. Lead 204A is situated as in FIGS. 7 and 8. Lead 204 has placements of thermal sensors, in addition to proximal sensor set 209 in the coronary sinus, one or more of the: 1) distal sensor set 206, located in the great cardiac vein distal to the place where the left marginal vein joins the great cardiac vein; 2) differential sensor set 207, 208 located in the course of the great cardiac vein to become the coronary sinus proximal to the place where the left marginal vein joins the great cardiac vein and distal to the place where the middle cardiac vein joins the coronary sinus.

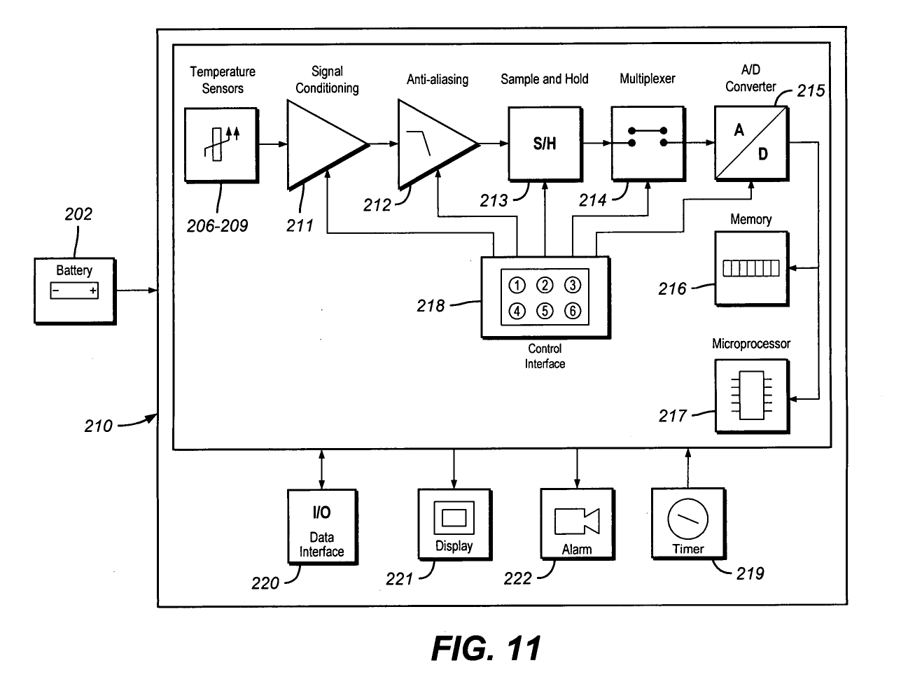

Referring to FIG. 11, a sensing and control system of this invention includes sensors such as thermal sensors 206-209, and a control unit 203 that includes, powered by battery 202, circuitry indicated generally by reference numeral 210 including components for signal conditioning 211, anti-aliasing 212, sample and hold 213, multi-plexing 214, and analog to digital conversion 215, the digital signals from 215 communicating in circuitry 210 with memory 216 and main processor 217. A control interface 218 allows human input to control unit 203, as for entering presets, as explained hereinbelow. Circuitry 210 receives signal inputs from timer 219 and data interface 220, and outputs signals to data interface 220, display 221 and alarm 222. Processor 217 i) clocks and calendars the activity of the device, employing input from timer 219; ii) connects through wires or wireless means to the sensor parts 206-209 and records data in memory at on a regular basis; iii) processes software codes; iv) communicates with the alarming components 222 and external input/output components 218, such as the sensor(s), keyboard or equivalent alphanumeric input device and monitor or equivalent alphanumeric output device 218; 2) communications connectors such as at 220, which connect the main processor to external input/output devices. The alarming process has two routes of output; the patient himself, and his health caregivers. I) The alarming for the patient might include one or more of: 1) sound signal; 2) voice signal (robotic message, prerecorded human voice; 3) vibratory alarm; and/or 4) visual alarm (blinking light). II) The alarming for the health caregiver might include one or more of: 1) automated call made to one or more of a set of predefined phone numbers; 2) wired or wireless communication with a closely located monitoring center.

Presetting the specificity and sensitivity for the alarming system customizes the alarming system toward the specific health condition and needs of the patient. The system alarms sooner if the patient is in an otherwise poorer prognostic category, suffers other mental/health disabilities, or is located away from healthcare. This presetting, which is done by the health care professional that oversees the functionality of the device, is based on 1) other prognostic factors; 2) patient's other co-morbidities; and 3) the availability of professional and/or nonprofessional healthcare. The said other prognostic factors: include older age, diabetes, CHD, previous myocardial infarction, Parkinson's disease. Presence of any of these would necessitate an increase in the preset sensitivity from the optimal point by 5%. The said other co-morbidities of a patient may include presence of i) Alzheimer's disease or other dementia, ii) depressive mood, and iii) motor disability. Presence of any of these items would direct the output the alarming system toward the healthcare provider. The said availability of professional and/or nonprofessional healthcare is categorized under 4 classes: A) immediately available (such as in the intensive care unit), B) available within minutes (such as in the hospital wards), C) available within 1 to 24 hrs (such as person living with healthy others close to medical centers in large cities), and D) available with more than 24 hr delay (such as person living alone in small cities). For example, category A may have a present sensitivity 10% lower than optimal, and category D may have a preset sensitivity 10% higher than optimal.

In order to correct for individual factors that affect measured temperatures, the invention method uses the mean, median and standard deviation of temperatures recorded in a time stamped and date stamped table to correct cutoff points with the patient's own baselines.

One embodiment of the present invention provides means of combining temperature with other prognostic factors for a combination prognostic score. Such other prognostic factors include one or more of: decreased coronary sinus /myocardial pH, decreased coronary sinus/myocardial pO₂, increased coronary sinus/myocardial pCO₂, increased coronary sinus/myocardial lactate, increased ratio of lactate to pyruvate in the coronary sinus/myocardium, increased ratio of the reduced form of nicotine amide adenine dinucleotide (NADH) to nicotine amide adenine dinucleotide (NAD⁺) in the coronary sinus/myocardium, increased ratio of the reduced form of nicotinamine-adenine dinucleotide phosphate (NADPH) to nicotinamine-adenine dinucleotide phosphate (NADPH) in the coronary sinus/myocardium, increased ST segment, decreased ST segment, ventricular tachycardia, T wave changes, QRS changes, decreased patient activity, increased respiratory rate, decreased transthoracic impedance, decreased cardiac output, increased pulmonary artery diastolic pressure, increased myocardial creatinine kinase, increased troponin, and changed myocardial wall motion. The parts that might provide the above mentioned information to the above said processing and alarming unit includes chest impedometer, devices that monitor the electrical activity, and devices that monitor patient's respiration, as known in the art.

The possibility of “random” changes in coronary sinus temperature over time (so called “noise” or “drift”), as well as changes related to heart rate and contractility is considered and corrected for in this invention. The right atrial temperature is helpful for reflecting general body activity, as is the addition of an accelerometer in a specific design of the invention. The right atrial temperature is also useful in patients who may be infected, exposed to heat or cold, etc.

For the changes in heart rate, rhythm and efficiency (e.g., suspect atrial fibrillation as an inefficient heart rhythm) or general coronary tone (i.e., cold-induced or anxiety-induced vasoconstriction), a specific design of this invention uses cardiac-specific baselines (baseline for the left ventricular heat production). Temperatures at the ostium of the great cardiac vein 19 and left marginal vein 24, in addition to the temperature in the coronary sinus 14, obtained as described in connection with FIG. 5, may be used for this specific purpose. In this way, the ischemia, or infarction, caused by reduction of flow in an area would be inferred not so much by relative temperature change compared to the right atrial temperature, but also compared to blood coming from areas supplied by the circumflex coronary artery and drained by the left marginal vein 24. A similar approach is chosen for detecting multiple lesions. Some patients with unstable angina and many patients with myocardial infarction have a second active lesion, and this is often in a different vessel. Differential temperature measurements along the course of coronary sinus will give information as to which area would have an abnormal temperature profile. Combining the information with that derived from the right atrium is, at any given moment, highly suggestive of which bed has an abnormal temperature.

In addition to changes in the temperature and flow, ischemia causes changes in the coronary sinus pressure wave, pO₂ and pH, as well as other indicators, which can be measured using sensors located on the tip of a lead positioned in the coronary sinus, such as sensor 206.

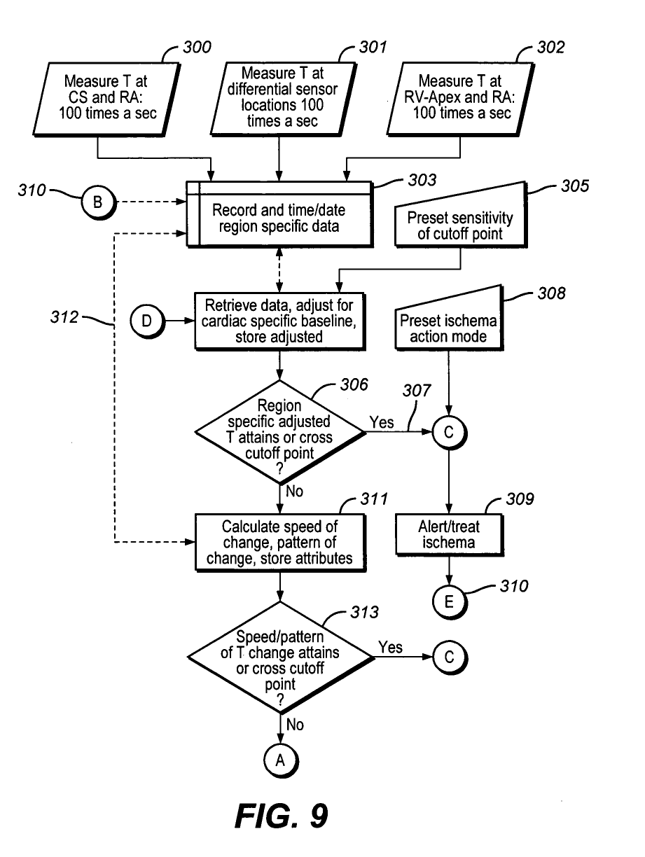

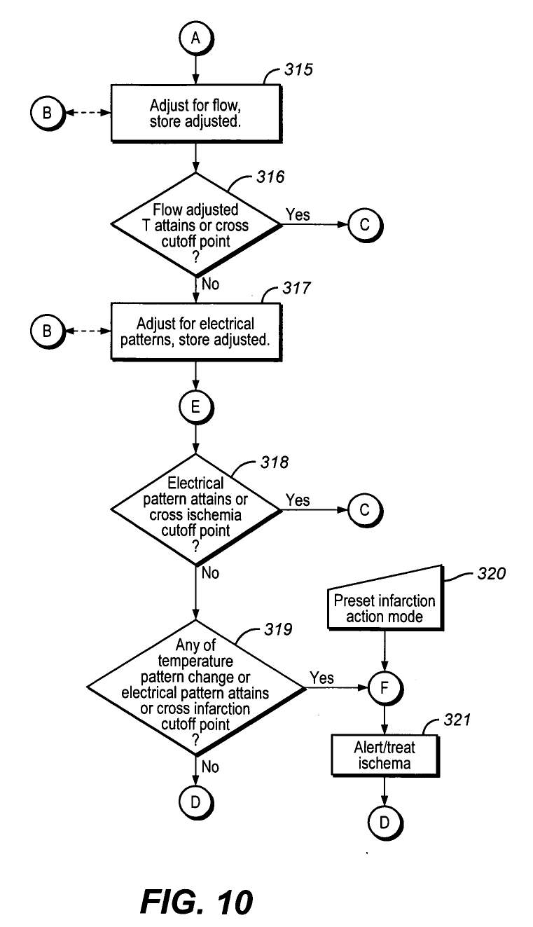

Referring to FIGS. 9 and 10, a flow path is illustrated showing a sequence of operations which can be performed by an algorithm incorporating options of the invention described above.

Processor 217 receives conditioned signals as explained from FIG. 11 from thermal sensors, for example, from sensors 206-209, positioned on leads 204 and 204A for location in the right atrium 10, the coronary sinus 14, at the right ventricular apex 28 and suitably with differentially in the great cardiac vein 19 as described in connection with FIG. 5. Thus measurements are taken at timing intervals in the coronary sinus and right atrium as at 300, in the great cardiac vein as at 301 and in the right ventricle apex and right atrium, as at 302. Receiving timing signals from timer 219, processor 217 records and time/dates at 303 the temperature sensor information received from the sensors measured as at 300, 301 and 302. The sampling rate shown in FIG. 9 is an example; sampling may be at a rate from once a minute to 1000/sec, more suitably in the range from about 30 per second to 240 per second.

Microprocessor 217 at 304 receives a preset sensitivity of cutoff point from 305, set using control interface 218. The factors set forth above concerning presets are used to set the preset sensitivity of the cutoff point.

Cardiac specific baseline data from point “D” of the flow path is received at 304, and temperature data recorded at 303 is retrieved and adjusted for the cardiac specific data. Each region specific temperature from 300, 301 and 303 as adjusted is checked against the cutoff point. If the temperature data as adjusted attains or crosses the cutoff point at 306, an output signal triggers “C” as at 307 and a preset ischemia action mode routine 308 energizes data interface 220, display 221 and/or alarm 222, to invoke preset ischemia action mode, as at as at 309. The action mode suitably may be an adjustment to the pacing of a pacemaker to a base line that does not over work the heart during the moments of ischemia, or to a defibrillator, or to a controller of an infusion pump for dispensing medication, or notification of health care personnel, or the like, as described hereinabove. The preset sensitivity of cutoff point signal is also sent to entry “E” of the flow path, as at 310.

If the temperature data as adjusted at 304 does not attain or cross the cutoff point at 306, microprocessor 217 calculates the speed of change or pattern of change or both, as at 311, writes the calculations to memory 216, as at 312, and determines as at 313, whether the speed and pattern of temperature change attains or crosses the cross over point. If it does an alert is triggered, invoking the preset ischemia action mode 308 for an alert and/or treatment of the ischemia as at 309. If at 313 speed and pattern of temperature change do not attain or cross the cross over point, referring to FIG. 10, a continuation of the process, the microprocessor receives timing information from timer 217 and flow data input at 315 from the sensors in the coronary sinus 14 and the monitored great cardiac vein 19 draining to the coronary sinus and adjusts the speed and pattern of change of temperature data from 313, according to a routine that incorporates the relationships of equation (3) or a similar suitable relationship. At 316, the microprocessor determines if the flow adjusted temperature attains or crosses the cutoff point. If so, routine “C” is invoked, described above. If not, the microprocessor adjusts the temperature data for electrical patterns and with a data stamp from timer 219 at “B” stores the adjusted data at 317. Processor 217 receives the preset ischemic action mode signal from “E” and tests to see if the electrical pattern attains or crosses the ischemic cutoff point, at 318. If so, routine “C” is invoked. If not, processor 217 checks to see if any of temperature pattern charge or electrical pattern attains or cross es an infarction cutoff point.. If so, a preset infarction action mode is routine 320 is triggered and action to expression of an alert and/or to treat the patient is triggered.

A. Applications of the Sensing and Control System and Variations in the Coronary Sinus

1. Pressure and Flow Changes In The Coronary Sinus Changes Following Myocardial Ischemia and infarction

As mentioned previously, in a specific design of this invention, flow in the coronary sinus may be measured, which can either be used as a standalone indicator of ischemia, or be used to further determine the extent of fall in H_{CS} as shown in (3), giving a better estimate of occurrence and extent of ischemia.

In still another specific design of this invention, pressure in the coronary sinus may be measured, which then again can further support diagnosis of ischemia. It has been previously described that following ischemia, pressure in the right ventricle increases, follows reduced contractility and the resulting increase in pulmonary artery pressure. Inventors deductively conclude that this causes an increase in the pressure in the right ventricle, which in turn causes an increase in the pressure in the coronary sinus. In previous experiences, pressure increase in the right ventricle preceded EKG changes in asymptomatic as well as symptomatic episodes of myocardia ischemia/infarction, which is expected to be the case for the coronary sinus pressure as well.

2. Coronary Sinus Blood Sampler

A coronary sinus catheter that has a lumen can be used to sample the molecular events in the coronary sinus in case the coronary sinus blood temperature or other indicator flags ischemia or infarction, or the patient develops symptoms or signs of such event. This could be a disposable catheter or a chronic implant with a subcutaneous diaphragm which could be punctured with a needle to draw blood. Of course, the initial 5 ml or so, plus the volume contained within the catheter, would be discarded.

Ideally, this would be a heparin-impregnated catheter or could be fitted with a small osmotic drug delivery system so as to deliver an anticoagulant (such as heparin, low-molecular-weight heparin), an antithrombin agent (such as Bivalirudin or Argatroban) or an antiplatelet agent (such as clopidogrel or EDTA).

Such a device obviously could be used for delivering drugs by injection (through the skin and diaphragm, which would be located in the region of the subclavian or in the neck over the internal jugular vein.

In general, the coronary sinus probably can be used to better understand the etiology, pathogenesis and natural history of a wide variety of cardiac conditions and also can be used in the development of therapies, including new drugs, vaccines, genes and gene products and devices, both investigational and clinical uses (e.g., tailoring therapy to the individual based on the initial measurements and then subsequently monitoring therapy to determine success, side effects and completion of therapy). In essence, it is a ‘biopsy’ of products released from the heart and products taken up or not taken up, so, for example, it can be used to study the kinetics, distribution and metabolism of drugs administered to the heart (by mouth, vein or intracoronary injection, pericardial or transcutaneous administration, or fits on a drug-eluting stent, etc.) Other therapies which could be studied, evaluated, monitored, etc., include stem cells and genetically modified autologous cells. The effects of diet can also be examined.

The coronary sinus can be used to analyze soluble components and particular components, such as cells and pieces of cells (e.g., apoptotic bodies). Examples of conditions that might be studied include coronary atherosclerosis, including coronary inflammation, plaque rupture, erosion and spasm and thrombosis, heart failure, congenital abnormalities, arrhythmias and hypertension and the results of surgery.

Variables which might be studied include matrix metalloproteases, heat-shock proteins, troponin, creatinine kinase, isoenzymes, adenosine, growth factors, endothelin, angiotensin-2, natriuretic peptides, hormones, oligonucleotides, cell-surface markers such as, annexin-5 for study of apoptosis or von Willebrand's factor. The DNA analysis can be used to look for acquired (somatic) mutations, as well as to determine the efficacy of gene transfer (i.e., gene therapy).

The coronary sinus can also be used to look for evidence of infection. In cells harvested from coronary sinus, calcium transients can be studied and the cells can be cultured for a wide variety of purposes. Membranes can also be used to study receptor binding. In general the coronary sinus approach has certain advantages over repetitive molecular imaging, such as reduced background and reduced cost and radioactivity, etc. The sampling might be useful in measuring levels of platelet-release products and other markers of thrombosis, including but not limited to beta thromboglobulin, fibrinopeptide A, and D-dimer. Also of interest are markers of inflammation, including VCAM-1, ICAM-1, MCP-1, IL-1, IL-6, IL-10, IL-18, activated T cells, TNF alpha, interferon gamma and others. Other analyses that might be important include markers of oxidation, such as oxidized LDL cholesterol and NO—SH compounds, including nitrosylated proteins. There is also utility in detecting aggregates of platelets and leukocytes and in detecting senescent endothelial cells, macrophages and massed cells.

This specific design is not an outpatient product, but, like the Hickman line and related lines used in cancer patients on chemotherapy, there may be a group of patients sent home on novel anti-inflammatory agents where it is important to discontinue them as soon as the plaques have quieted down. In cases like this, temperature may not be a sufficient marker.

More likely, this catheter might be useful in research protocols to assist pharmaceutical companies developing novel medications.

3. Coronary Sinus Thermal Analysis for Use in Temporary Diagnostic Settings

The analysis of coronary sinus blood temperature and the way it would change in response to a variety of stimulations is useful in determination of the following:

a) Presence and adequacy of collateral flow in presence of a suspicious atherosclerotic lesion in the coronary arterial tree. . This can be done in a temporary application of the invention device, as well as the implantable one, in the setting of a cardia catheterization laboratory, where a balloon is gently passed over a suspect atherosclerotic lesion and would be gently inflated for a short period of time to the extent to completely obstruct the flow, but not cause endothelial injury of stretch the arterial wall. The response of either of coronary sinus blood temperature, or right ventricular apex myocardial temperature, or both, to occlusion determines if sufficient collateral circulation exists, in which coronary sinus temperature changes would be minimal, or rapidly recovering. If coronary sinus temperature changes would be indicative of ischemia, or take a relatively long time to recover, it would be indicative of insufficient collateral circulation, in which case therapy would be indicated.

b) Assessment of coronary artery endothelial dysfunction in presence of a variety of disease, including but not limited to hypertensive heart diseases, hypertrophic cardiomyopathy, and diabetes mellitus. This can be done in a temporary application of the invention device, as well as the implantable one, in the setting of a cardia catheterization laboratory, where an infusion of the endothelium-dependent vasodilator acetylcholine into the left coronary artery of a suspect patient will be done. If coronary arteries respond to the vasodilator medication, the temperature in coronary sinus will decrease. If such change would not happen, an impairment of endothelium-dependent dilation of coronary arteries, which in turn puts the patient in higher risk category for ischemic events, and would justify appropriate treatment, which may include L-arginine supplementation and other more aggressive means.

c) Assessment of coronary artery endothelium-independent impaired vasodilation as a result of aging, hypertension and a variety of other diseases such as diabetes mellitus. This can be done in a temporary application of the invention device, as well as the implantable one, in the setting of a cardia catheterization laboratory, where an infusion of the endothelium-independent vasodilators papaverine, and glyceryl trinitrate, or isosorbide dinitrate into the left coronary artery of a suspect patient will be done. If coronary arteries respond to the vasodilator medication, the temperature in coronary sinus will decrease. If such change would not happen, an impairment of endothelium-dependent dilation of coronary arteries, which in turn puts the patient in higher risk category for ischemic events, and would justify appropriate treatment.

B. Applications of the Sensing and Control System and Variations in the Right Ventricular Apex

1. Myocardial Temperature, Pressure, Electrogram, pO2 and pH in the Right Ventricular Apex Change Following Myocardial Ischemia

In a specific design of this invention, a coronary sinus sensor is placed in such a way as to actually contact the wall and therefore reflect the myocardial temperature. Clearly, the best place for measurement of myocardial temperature in order to detect myocardial ischemia is at the RV apex, since that is the watershed zone for ischemia and is most sensitive. In situations when justification is not available for having an electrode in the RV apex plus one in the coronary sinus, this alone sensor might help in determining myocardial temperature.

Diagnosis of myocardial ischemia in the area of the apex of the heart poses difficulty because of the insensitivity of electrocardiogram. In this invention, measurement of regional temperature in the apex is used to detect ischemia in that region. Using sensors that are embedded in myocardial tissue in the right ventricular apex by either wedged them under a trabeculum or screwing them in the tissue like pacemaker leads; it is possible to measure temperature, pressure, electrogram, tissue pO2 and pH, as well as several other factors, and these will give more indicators for myocardial ischemia and infarction.

Using infrared imaging to assess regional temperature changes, as well other means of measuring local epicardial temperature in animal and human models of myocardial ischemia, it has been established for several decades now that upon closure of the left main coronary artery in the heart, regional temperature in the ischemic area decreases. In addition to changes in the temperature, local pressure drops because of the loss of contractility and dyskinesis of the involved myocardium. Also, local pO₂ and pH decreases because of the metabolic disturbance in the area. There will also be changes in the electrogram (EGM) sensed by local sensor, and all these are useful in determining the occurrence of ischemia and/or infaction.

2. Temperature Adjustment for Electrogram of the Right Ventricular Apex Improves Detection of Myocardial Ischemia

Right ventricular apex electrogram is useful in detection of myocardial ischemia. However, local temperature changes affect electrogram readings. This invention provides a temperature sensor in the same area where electrogram is being recorded. This enables a correction algorithm to adjust the signal recorded from the intramyocardial electrogram with the temperature of that location, and therefore increases the accuracy of detection of ischemia.

3. Addition of Continuous Electrocardiogram Improves Detection of Myocardial Ischemia

The design of the device subject of this invention makes it possible to record continuous electrogram using electrodes on either of the catheters that go into coronary sinus or right ventricular apex and connect to an implantable box. One electrode is placed on the box and in contact with tissue. A second electrode is placed on the shaft of the catheter in touch with blood stream in superior vena cava just superior to right atrium, and a third electrode is placed on the shaft of the catheter close to the tip right proximal to the temperature sensor. In either of the coronary sinus catheter or right ventricular catheter designs, the placement of the above said electrodes would form a triangle around the heart. Such positioning would make continuous electrocardiogram recording, which enhances diagnosis of myocardial ischemia.

References

(Inclusion of Articles, Patents or Published Patent Applications in this Listing is not an Acknowledgment That They Constitute Prior Art.)

Issued Patents

1. U.S. Pat. No. 4,681,117, commonly invented by Brodman; Richard F. (3388 Wayne Ave., Bronx, N.Y. 10467); Siegel; Sharon B. (3450 Wayne Ave., Bronx, N.Y. 10467), issued Jul. 21, 1987, entitled “Intracardiac catheter and a method for detecting myocardial ischemia”.

2. U.S. Pat. No. 5,135,004, commonly invented by Adams; John M. (Issaquah, Wash.); Alfemess; Clifton A. (Redmond, Wash.), issued Aug. 4, 1992, entitled “Implantable myocardial ischemia monitor and related method”.

3. U.S. Pat. No. 5,199,428, commonly invented by Obel; Israel W. P. (Johannesburg, ZA); Bourgeois; Ivan (Verviers, BE), issued Apr. 6, 1993, entitled “Implantable electrical nerve stimulator/pacemaker with ischemia for decreasing cardiac workload”.

4. U.S. Pat. No. 6,021,350, invented by Mathson; Goran (Uppsala, SE), issued Feb. 1, 2000, entitled “Implantable heart stimulator with a maximum stimulation rate that is adjusted dependent on ischemia detection”.

5. U.S. Pat. No. 6,112,116, commonly invented by Fischell; Robert E. (Dayton, Md.); Fischell; David R. (Fair Haven, N.J.); Fischell; Tim A. (Richland, Mich.), issued Aug. 29, 2000, entitled “Implantable responsive system for sensing and treating acute myocardial infarction”.

6. U.S. Pat. No. 6,128,526, commonly invented by Stadler; Robert (Shoreview, Minn.); Nelson; Shannon (Stacy, Minn.); Stylos; Lee (Stillwater, Minn.); Sheldon; Todd J. (Eagan, Minn.), issued Oct. 3, 2000, entitled “Methods for ischemia detection and apparatus for using same”.

7. U.S. Pat. No. 6,238,422, invented by Van Oort; Geeske (Nieuwleusen, NL), issued May 29, 2001, entitled “Pacemaker system with therapy for minimizing risk of morning myocardial infarctions or arrhythmias”.

8. U.S. Pat. No. 6,243,603, commonly invented by Ideker; Raymond E. (Birmingham, Ala.); KenKnight; Bruce H. (Maple Grove, Minn.), issued Jun. 5, 2001, entitled “Methods and apparatus for detecting medical conditions of the heart”.

9. U.S. Pat. No. 6,368,284, invented by Bardy; Gust H. (Seattle, Wash.), issued Apr. 9, 2002, entitled “Automated collection and analysis patient care system and method for diagnosing and monitoring myocardial ischemia and outcomes thereof”.

10. U.S. Pat. No. 6,501,983, commonly invented by Natarajan; Ananth (New Port Richey, Fla.); Thakor; Nitish V. (Clarksville, Md.), issued Dec. 31, 2002, entitled “Implantable myocardial ischemia detection, indication and action technology”.

Patent Applications

11. U.S. patent application Document Number 20020072777, invented by Lu, Richard (Thousand Oaks, Calif.), published Jun. 13, 2002, entitled “Method and device for responding to the detection of ischemia in cardiac tissue”.

12. U.S. patent application Document Number 20020143262, invented by Bardy, Gust H. (Seattle, Wash.), published Oct. 3, 2002, entitled “System and method for providing diagnosis and monitoring of myocardial ischemia for use in automated patient care”.

13. U.S. patent application Document Number 20030125774, invented by Salo, Richard (Fridley, Minn.), published Jul. 3, 2003, entitled “Method and apparatus for monitoring left ventricle work and power”.

14. U.S. patent application Document Number 20030130581, invented by Salo, Richard (Fridley, Minn.), published Jul. 10, 2003, entitled “Method and apparatus for measuring left ventricle pressure”.

15. U.S. patent application Document Number 20030167081, invented by Zhu, Q. et al (Little Canada, Minn.), published Jul. 3, 2003, entitled “Coronary sinus lead with thermal sensor and method therefor”.

Other References

16. Stewart J T, Gray H H, Calicott C, Smith R E, Camm A J. Heat production by the human left ventricle: measurement by a thermodilution technique. Cardiovascular Research, 1990, 24, 418-422

17. Henry Gray, Mary Dyson, P. L. Williams (Editor), Richard M. Warwick (Editor). Gray's Anatomy, 37th edition (1989), Churchill Livingstone Publishers. Pages 703 and 792-3.

18. Worley S J, “Demystifying the Coronary Sinus” Nov. 21, 2002, URL: http://www.bivtricks.org/demystifyingy_sinus.htm

19. Solomon R, Werner C, Mann D, D'Elia J, Silva P. Effects of saline, mannitol, and furosemide to prevent acute decreases in renal function induced by radiocontrast agents. N Engl J Med 1994; 331:1416-20

20. Parfrey P S, Griffiths S M, Barrett B J, Paul M D, Genge M, Withers J, Farid N, McManamon P J. Contrast material-induced renal failure in patients with diabetes mellitus, renal insufficiency, or both. A prospective controlled study N Engl J Med 1989; 320:143-9

21. Shyu K G, Cheng J J, Kuan P. Acetylcysteine protects against acute renal damage in patients with abnormal renal function undergoing a coronary procedure. J Am Coll Cardiol. 2002 Oct. 16;40(8):1383-8.

22. Tepel M, van der Giet M, Schwarzfeld C, Laufer U, Liermann D, Zidek W. Prevention of radiographic-contrast-agent-induced reductions in renal function by acetylcysteine. N Engl J Med. 2000 Jul. 20;343(3):180-4.

23. Vavouranakis M, Tsiamis E, Vaina S, Toutouzas K, Glalafos J, Boudoulas H, Toutouzas P, Statanadis C. Increased Coronary Sinus Temperature in Patients With Significant Atherosclerotic Lesions in Left Coronary Artery Determined by a New Technique: The Coronary Sinus Thermography. J Am Col Card Mar. 19, 2003, 14(6) 41A-42A

24. Chierchia S, Lazzari M, Freedman B, Brunelli C, Maseri A. Impairment of myocardial perfusion and function during painless myocardial ischemia. J Am Coll Cardiol 1983 March;1(3):924-30

25. Khomaziuk A I, Neshcheret A P, Sher L A. [A method of continuous registration of heat production of the heart] [Article in Russian] Biull Eksp Biol Med. 1988 July; 106(7): 124-6.

26. Sher L A, Khomaziuk A I, Neshcheret A P. [A device for the continuous recording of the temperature of the coronary venous blood] [Article in Russian] Fiziol Zh. 1988 July-August;34(4):114-6.

27. Stefanadis C, Tsiamis E, Vaina S, Toutouzas K, Boudoulas H, Gialafos J, Toutouzas P. Temperature of blood in the coronary sinus and right atrium in patients with and without coronary artery disease. Am J Cardiol. 2004 Jan. 15;93(2):207-10.

1. A method of detecting cardiac ischemia comprising placing a temperature sensor in the coronary sinus of a heart and taking a blood core temperature (T_{C}) elsewhere, measuring temperature in the coronary sinus (T_{CS}) sensed by said sensor in the coronary sinus and determining if T_{CS} increases relative to T_{C}.

2. A method of detecting cardiac ischemia comprising placing a temperature sensor in the coronary sinus of a heart after taking a baseline temperature (T-baseline) in the right atrium of the heart with said sensor, measuring temperature in the coronary sinus (T_{CS}) sensed by said sensor in the coronary sinus and determining if T_{CS} increases relative to T-baseline.

3. A method of detecting cardiac ischemia comprising placing a temperature sensor in the coronary sinus of a heart and a temperature sensor in the right atrium of a heart, measuring temperature in the coronary sinus (T_{CS}) sensed by said sensor in the coronary sinus and measuring temperature in the right atrium (T_{RA}) sensed by said sensor in the right atrium and determining if T_{CS} increases relative to TRA.

4. A method of detecting cardiac ischemia comprising attaching a temperature sensor in the wall of the apex of a heart, taking a blood core temperature (T_{C}) elsewhere, measuring temperature of the apex (T-apex) sensed by said sensor in said wall and determining if T-apex decreases relative to T_{C}.

5. A method of detecting cardiac ischemia comprising attaching a temperature sensor in the wall of the apex of a heart after taking a baseline temperature (T-baseline) in the right atrium of the heart with said sensor, measuring temperature of the apex (T-apex) sensed by said sensor in said wall and determining if T-apex decreases relative to T-baseline.

6. A method of detecting cardiac ischemia comprising attaching a temperature sensor in the wall of the apex of a heart and a temperature sensor in the right atrium of a heart, measuring temperature of the apex (T-apex) sensed by said sensor in said wall and measuring temperature in the right atrium (T_{RA}) sensed by said sensor in the right atrium and determining if T-apex decreases relative to T_{RA}.

7. A method of detecting cardiac ischemia comprising placing a temperature sensor in the coronary sinus of a heart, taking a blood core temperature (T_{C}) elsewhere, attaching a temperature sensor in the wall of the apex of a heart, measuring temperature in the coronary sinus (T_{CS}) sensed by said sensor in the coronary sinus, measuring temperature of the apex (T-apex) sensed by said sensor in said wall, and determining if the ratio of T_{CS} to T-apex increases.

8. A method of detecting cardiac ischemia comprising placing a temperature sensor in the coronary sinus of a heart after taking a baseline temperature (T-baseline) in the right atrium of the heart with said sensor, attaching a temperature sensor in the wall of the apex of a heart, measuring temperature in the coronary sinus (T_{CS}) sensed by said sensor in the coronary sinus, measuring temperature of the apex (T-apex) sensed by said sensor in said wall, and determining if and determining if the ratio of T_{CS} to T-apex increases.

9. A method of detecting cardiac ischemia comprising placing a temperature sensor in the coronary sinus of a heart and a temperature sensor in the right atrium of a heart, attaching a temperature sensor in the wall of the apex of a heart, measuring temperature in the coronary sinus (T_{CS}) sensed by said sensor in the coronary sinus, measuring temperature of the apex (T-apex) sensed by said sensor in said wall, and determining if the ratio of T_{CS} to T-apex increases.

10. A method of detecting cardiac ischemia comprising placing a temperature sensor in the coronary sinus of a heart, measuring the waveforms of temperature in the coronary sinus (T_{CS}) sensed by said sensor in the coronary sinus relative to a core blood temperature baseline and determining if the average of the waveforms increases.

11. The method of claim 10 further characterized by determining if the average increased waveform is characterized by a rapid slope of increase slowing to a plateau.

12. The method of claim 10 further characterized in that said average is the mean of the oscillation of the waveform.

13. The method of claim 10 further characterized in that said average is the size of the area of the curve under the waveform.

14. The method of claim 10 in which said core temperature baseline is taken by said sensor in the right atrium of the heart before placing the sensor in the coronary sinus.

15. The method of claim 10 further comprising placing a temperature sensor in the right atrium of the heart and measuring temperature in the right atrium (T_{RA}) sensed by said sensor in the right atrium to establish baseline core temperature.

16. The method of claim 10 further comprising determining the size and/or severity of a region of ischemia by determining the increase of average of the waveforms before cessation of an event of ischemia.

17. The method of claim 10 further comprising determining cessation of an event of ischemia by determining when the waveform average decreases after said increase, to values below pre-ischemic values.

18. The method of claim 10 further comprising determining cessation of an event of ischemia by determining that the slope of decrease of the average of the waveform is sharper than the slope of increase of the waveform.

19. A method of detecting cardiac ischemia involving tachycardia comprising placing a temperature sensor in the coronary sinus of a heart, measuring the waveforms of temperature in the coronary sinus (T_{CS}) sensed by said sensor in the coronary sinus relative to a core blood temperature baseline and determining if the frequency of the waveforms increases.

20. The method of claim 19 further comprising determining the size and/or severity of a region of ischemia by determining an increase of frequency the waveforms before cessation of an event of ischemia.

21. The method of claim 19 comprising determining cessation of an event of ischemia by determining when the waveform frequency decreases after said increase to pre-ischemic values.

22. The method of claim 19 in which said core temperature baseline is taken by said sensor in the right atrium of the heart before placing the sensor in the coronary sinus.

23. The method of claim 19 further comprising placing a temperature sensor in the right atrium of the heart and measuring temperature in the right atrium (T_{RA}) sensed by said sensor in the right atrium to establish baseline core temperature.

24. A method of detecting cardiac ischemia comprising placing a temperature sensor in the coronary sinus of a heart, measuring the waveforms of temperature in the coronary sinus (T_{CS}) sensed by said sensor in the coronary sinus relative to a core blood temperature baseline, and determining if the amplitude of the waveforms decreases.

25. The method of claim 24 further comprising determining the size and/or severity of a region of ischemia by determining the decrease of amplitude of the waveforms.

26. The method of claim 24 in which said core temperature baseline is taken by said sensor in the right atrium of the heart before placing the sensor in the coronary sinus.

27. The method of claim 24 further comprising placing a temperature sensor in the right atrium of the heart and measuring temperature in the right atrium (T_{RA}) sensed by said sensor in the right atrium to establish baseline core temperature.

28. A method of detecting rhythm abnormality of the heart comprising placing a temperature sensor in the coronary sinus of a heart, measuring the waveforms of temperature in the coronary sinus (T_{CS}) sensed by said sensor in the coronary sinus relative to a core blood temperature baseline, and determining if the waveforms of temperature in the coronary sinus is atypical for waveforms of temperature in the normal heart.

29. A method of tracing pressure in coronary arteries comprising placing a temperature sensor in the coronary sinus of a heart, measuring the waveforms of temperature in the coronary sinus (T_{CS}) sensed by said sensor in the coronary sinus relative to a core blood temperature baseline, and ascertaining the phase of said waveforms.

30. A method of determining blood flow in coronary arteries comprising placing a temperature sensor in the coronary sinus of a heart, measuring the waveforms of temperature in the coronary sinus (T_{CS}) sensed by said sensor in the coronary sinus relative to a core blood temperature baseline, and ascertaining the shape of said waveforms.

31. A method of monitoring a patient with coronary artery disease for occurrence of myocardial ischemia and/or infarction, which:
(a) measures patient's core body temperature,
(b) measures patient's right ventricular apex myocardial temperature,
(c) calculates their difference,
(c) uses the above said to indicate a change in the temperature gradient as criteria to indicate occurrence of ischemia and/or infarction.
(d) selectively alarms the patient or the health caregiver in a time that best suits patient's condition.

32. The method of claim 31 that is further characterized by that to indicate ischemia and/or infarction, it analyzes the characteristics of the pattern of temperature change across the coronary sinus and the great cardiac vein at two or more locations.

33. The method of claim 33 that is further characterized by that data from two or more locations across the coronary sinus and the great cardiac vein is used to indicate the specific region of the heart that suffers ischemia and/or infarction.

34. The method of claim 31 or 32 that is further characterized in that to indicate ischemia and/or infarction, the above said criteria would be adjusted based on the desired sensitivity of the alarming algorithm.

35. The method of claim 31 or 32 that is further characterized in that to indicate ischemia, it uses the above said in addition to any one or more of the following internal and external factors that affect patient's body temperature and the activity of the heart:
(a) patient's level of activity
(b) use of medications, alcohol consumption, and smoking

36. The method of claim 31 in which said temperature change will be corrected for coronary sinus flow.

37. The method of claim 31 in which information regarding one or more of the following factors will be analyzed to indicate the occurrence of myocardial ischemia and/or infarction: increased coronary sinus pressure, decreased coronary sinus pH, decreased coronary sinus pO2, increased coronary sinus pCO2, increased coronary sinus lactate, increased ratio of lactate to pyruvate in the coronary sinus, increased ratio of the reduced form of nicotine amide adenine dinucleotide (NADH) to nicotine amide adenine dinucleotide (NAD+) in the coronary sinus, increased ratio of the reduced form of nicotinamine-adenine dinucleotide phosphate (NADPH) to nicotinamine-adenine dinucleotide phosphate (NADPH) in the coronary sinus, increased ST segment, decreased ST segment, ventricular tachycardia, T wave changes, QRS changes, decreased patient activity, increased respiratory rate, decreased transthoracic impedance, decreased cardiac output, increased pulmonary artery diastolic pressure, increased myocardial creatinine kinase, increased troponin, and changed myocardial wall motion.

38. The method of claim 32 in which information regarding one or more of the following factors will be analyzed to indicate the occurrence of myocardial ischemia and/or infarction: decreased local myocardial pressure, decreased myocardial pH, decreased myocardial pO2, increased myocardial pCO2, increased myocardial lactate, increased ratio of lactate to pyruvate in the myocardium, increased ratio of the reduced form of nicotine amide adenine dinucleotide (NADH) to nicotine amide adenine dinucleotide (NAD⁺) in the myocardium, increased ratio of the reduced form of nicotinamine-adenine dinucleotide phosphate (NADPH) to nicotinamine-adenine dinucleotide phosphate (NADPH) in the myocardium, increased ST segment, decreased ST segment, ventricular tachycardia, T wave changes, QRS changes, decreased patient activity, increased respiratory rate, decreased transthoracic impedance, decreased cardiac output, increased pulmonary artery diastolic pressure, increased myocardial creatinine kinase, increased troponin, and changed myocardial wall motion.

39. Apparatus and software for monitoring a patient with coronary artery disease and indicate occurrence of myocardial ischemia and/or infarction, comprising:
(a) rewritable data storage that keeps patients customized alarming criteria, and other information detailed below,
(b) two temperature detectors for sensing temperatures of a patient's right atrium and coronary sinus and generating signals representative of the sensed temperatures,
(c) output device providing means for alarming the occurrence of myocardial ischemia and/or infarction,
(d) processing unit that:
(i) communicates with temperature sensors and rewritable data storage
(ii) runs the software that records temperature measurements, calculates the gradient and its change over time and analyses the pattern of temperature change based on the output of (b), stores these on a time stamped basis in (a), and uses such to detect occurrence of myocardial ischemia and/or infarction.
(iii) communicates with the output device (c) to alarm the patient or patient's health caregiver.

40. The apparatus of claim 40 in which said detectors are located in the right atrium and right ventricular apex.
